(19) United States
(12) Patent Application Publication
(10) Pub. No.: US 2021/0052582 A1
(43) Pub. Date: Feb. 25, 2021

(54) METHODS OF USE AND PHARMACEUTICAL COMPOSITIONS OF A SELECTIVE SYK INHIBITOR

(71) Applicants: Portola Pharmaceuticals, Inc., South San Francisco, CA (US); ORA, Inc., Andover, MA (US)

(72) Inventors: Anjali PANDEY, San Francisco, CA (US); Matthew CHAPIN, Andover, MA (US); Harold PATTERSON, Andover, MA (US); Yung Yueh HSU, Andover, MA (US); Mark ABELSON, Andover, MA (US)

(73) Assignees: Portola Pharmaceuticals, Inc., South San Francisco, CA (US); ORA, Inc., Andover, MA (US)

(21) Appl. No.: 16/979,075

(22) PCT Filed: Mar. 8, 2019
(86) PCT No.: PCT/US2019/021402

§ 371 Date: Sep. 8, 2020

Related U.S. Application Data
(60) Provisional application No. 62/663,999, filed on Apr. 27, 2018.
(60) Provisional application No. 62/641,094, filed on Mar. 9, 2018.

(51) Int. Cl. A61K 31/501; A61K 9/00; A61P 27/14; A61K 47/10; A61K 47/18

(52) U.S. Cl. CPC A61K 31/501; A61K 9/0048; A61K 47/18; A61K 47/10; A61P 27/14

(57) ABSTRACT

Provided herein are methods of using Syk inhibitors, such as a selective Syk inhibitor, Compound 1 or a pharmaceutically acceptable salt thereof, in treating allergic and/or inflammatory diseases or conditions of the eye. Also provided is pharmaceutical compositions, in particular eyedrop ophthalmic compositions, comprising Compound 1 or a pharmaceutically acceptable salt thereof, useful in the methods.

### CROSS-REFERENCE TO RELATED APPLICATIONS

This application claims the benefit of U.S. Provisional Application Nos. 62/641,094, filed Mar. 9, 2018 and 62/663,999, filed Apr. 27, 2018, which is hereby incorporated by reference in its entirety.

### FIELD

This disclosure relates to methods of treating ophthalmic diseases, such as allergic conjunctivitis and inflammatory diseases of the eye, and pharmaceutical compositions useful in the methods.

### BACKGROUND

Millions of Americans suffer from eye allergies. Most approved treatments for eye allergies are antihistamines, mast cell stabilizers, or both, and these drugs act primarily to reduce the signs and/or symptoms of the early phase allergic reaction. Traditional mast cell stabilizers have limited efficacy. Drugs with anti-histamine activity can work more acutely, and during the acute phase of the allergic reaction, but work generally more on itching, than on redness or swelling. While efficacious, anti-histamines and antihistaine/mast-cell stabilizers do not fully reduce both signs and symptoms, and a large portion of patients are not completely satisfied with their relief. Steroids are also used for more severe cases, but generally have limited efficacy dosed in an acute fashion, need to be dosed over time, and have side effects when dosed chronically as topical ocular eyedrops. New treatment options are needed that have rapid onset of action, long duration of action, are better at treating signs and symptoms, and are safer with repeat dosing. There is also evidence that suggests that many eye allergy patients exhibit a persistent late inflammatory response needing anti-allergy medications that are effective not only in the treatment of the acute allergic reaction, but also of the more complex chronic inflammatory environment that results from overlapping and continual allergen exposure. Existing treatments available on the market do not sufficiently address the persistent or ongoing allergic reaction or inflammatory component of the reaction.

Dry eye disease is a relatively common condition characterized by inadequate tear film protection of the cornea. Dry eye symptoms have traditionally been managed with eyelid hygiene, topical antibiotics (erythromycin or bacitracin ointments), oral tetracyclines (tetracycline, doxycycline, or minocycline), anti-inflammatory compounds (cyclosporine) and corticosteroids which are often time consuming, frustrating, and frequently ineffective or variably effective treatments. Tens of millions of people are affected worldwide by dry eye, and nearly five million Americans 50 years of age and older are estimated to have dry eye. Of these, more than three million are women and more than one and a half million are men. Elderly people frequently experience dryness of the eyes, but dry eye can occur at any age. Dry eye is also environmental and can be caused by extended visual tasking as well. Dry eye is a potentially disabling disease adversely impacting the vision-related quality of life. Current therapeutic options are limited and costly. Despite the high incidence of dry eye disease, it still remains a therapeutic challenge. Accordingly, there remains a need for new therapies to treat dry eye disease.

Dry eye, also referred to as keratoconjunctivitis sicca (KCS), can be a temporary or chronic condition. Severe dry eye is a debilitating disease that affects millions of patients worldwide and can cripple some patients. Millions of these individuals suffer from the most severe form. This disease often inflicts severe ocular discomfort, results in a dramatic shift in quality of life, induces poor ocular surface health, substantially reduces visual acuity and can threaten vision. Patients with severe dry eye develop a sensitivity to light and wind that prevents substantial time spent outdoors, and they often cannot read or drive because of the discomfort.

Beyond allergy, there is a need for novel anti-inflammatory agents for treating ocular diseases and conditions. Currently therapies such as steroids, have well known ocular side effects when dosed repeatedly for sustained periods of times (e.g. more than several weeks). Thus, there is a need for treatments which are as effective or more effective, and/or safer than existing anti-inflammatory agents.

### SUMMARY

Provided herein are methods of using a Syk inhibitor in the treatment of ophthalmic allergic, dry eye, and/or inflammatory diseases. In some embodiments, provided is a method of treating an ophthalmic disease comprising administering a therapeutically effective amount of a Syk inhibitor topically to an eye of a patient in need thereof.

In some embodiments, provided is a method of using 2-((1R,2S)-2-aminocyclohexylamino)-4-(3-(pyrimidin-2-yl)phenylamino)pyrimidine-5-carboxamide, a specific Syk inhibitor, or a salt thereof, in the treatment of ophthalmic diseases.

2-((1R,2S)-2-aminocyclohexylamino)-4-(3-(pyrimidin-2-yl)phenylamino)pyrimidine-5-carboxamide (herein also referred to as Compound 1) is of the formula:

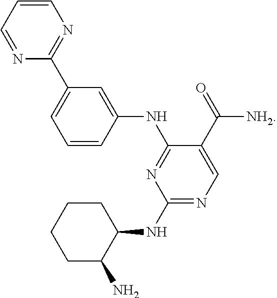 [SMILES: NC(=O)c1cnc(N[C@@H]2CCCC[C@@H]2N)nc1Nc1cccc(-c2ncccn2)c1]

It is described in U.S. Pat. No. 8,318,755, which is incorporated by reference in its entirety.

In some embodiments, provided herein is a method for treating an ophthalmic disease or condition comprising administering to a patient in need thereof a therapeutically effective amount of Compound 1 or a pharmaceutically acceptable salt thereof.

In some embodiments, the ophthalmic disease or condition is allergic and/or inflammatory, including one or more of allergic conjunctivitis (also called ocular allergy or eye allergy, including acute allergic conjunctivitis, chronic allergic conjunctivitis, temporary allergic conjunctivitis, persistent allergic conjunctivitis, seasonal allergic conjunctivitis or perennial allergic conjunctivitis), rhinoconjunctivis, dry eye, keratoconjunctivitis, eye inflammation, inflammation of the ocular surface or eyelids (e.g., dry eye, blepharitis, ptyergium, peripheral corneal infiltrate, post corneal transplant, pingueculitis, episcleritis, scleritis, keratitis, fungal keratitis, dermatitis of the eyelids, bacterial and viral conjunctivitis, and atopic keratoconjunctivitis (AKC), neurotrophic keratitis, GVHD-graft versus host disease), other ocular surface inflammation, irritation, and/or hyperemia, and/or anterior chamber of the eye (e.g., anterior uveitis, post-operative inflammation, iritis), vernal keratoconjunctivitis (VKC), giant papillary conjunctivitis (GPC), neurotrophic keratitis, GVHD-graft versus host disease, traumatic and post-surgical iritis, uveitis, pingueculum, pterygium, contact lens induced dry eye, other steroid-responsive inflammation of the palpebral and bulbar conjunctiva, cornea, and anterior segment of the globe, posterior uveitis, retina diseases such as macular edema associated with cystoid macular edema, diabetic macular edema, branch retinal vein occlusion (BRVO), central retinal vein occlusion (CRVO), eye redness, swollen eye/chemosis, eyelid swelling, eyelid congestion, and itchy eye, steroid-responsive inflammation of the palpebral and bulbar conjunctiva, cornea, and anterior segment of the globe, and ocular conditions for which a corticosteroid is indicated. In some embodiments, the ophthalmic disease or condition is acute or chronic allergic conjunctivitis, which may be seasonal, perennial, temporary, or persistent allergic conjunctivitis.

In some embodiments, the methods treat an anterior segment inflammatory disease. In some embodiments, the methods treat an ocular surface inflammatory disease, such as dry eye, blepharitis, ptyergium, peripheral corneal infiltrate, post corneal transplant, pingueculitis, episcleritis, scleritis, atopic keratoconjunctivitis, vernal keratoconjunctivitis, fungal keratitis (via effect of TLR signaling), bacterial or viral conjunctivitis (treating the inflammatory component—not necessarily as an anti-infective), steroid-responsive inflammation of the palpebral and bulbar conjunctiva, cornea, and anterior segment of the globe, and other ocular conditions for which a corticosteroid is indicated. In some embodiments, the methods treat an anterior chamber inflammatory disease, such as anterior uveitis, post-operative inflammation, or traumatic and post-surgical iritis.

In some embodiments, the methods treat one or more signs/symptoms of allergic conjunctivitis, including redness, itchiness, eyelid swelling, conjunctival swelling, discomfort, watery eyes, sensitivity to light, keratitis, corneal staining, conjunctival staining, or markers of inflammation of the eye, etc. In some embodiments, the methods treat one or more signs and/or symptoms of dry eye including, discomfort, dryness, grittiness, dryness, burning, keratitis, conjunctival redness, conjunctival staining, corneal staining, reduced tearing, reduced tear film break up time, reduced quality of life, reduced visual function.

In some embodiments, the methods treat dry eye.

In some embodiments, provided herein is a method for treating an ophthalmic disease or condition comprising administering topically to a patient in need thereof about 0.001 mg to about 1 mg of Compound 1 or a pharmaceutically acceptable salt thereof once a day, twice a day, three times a day, or four times a day. In some embodiments, the method comprises administering about 0.001 mg to about 1 mg of Compound 1 or a pharmaceutically acceptable salt thereof to each eye of the patient.

In some embodiments, provided are pharmaceutical compositions, specifically ophthalmic compositions in the form of eyedrops, comprising Compound 1, or a pharmaceutically acceptable salt thereof, and a pharmaceutically acceptable vehicle, suitable for treating ophthalmic diseases or conditions.

In some embodiments, provided herein are eyedrop ophthalmic compositions comprising Compound 1 or a pharmaceutically acceptable salt thereof, a buffer, a tonicity modifier, and a vehicle such as water. In some embodiments, the eyedrop ophthalmic compositions further comprise a preservative. In some embodiments, the eyedrop ophthalmic composition further comprise a demulcent, surfactant, or polymer system.

The eyedrop ophthalmic compositions described herein can comprise Compound 1 or a pharmaceutically acceptable salt thereof in an amount of about 0.001% to about 10%; about 0.01% to about 10%; about 0.05% to about 10%; about 0.1% to about 10%; about 0.2% to about 7%; about 0.3% to about 5%; about 0.4% to about 2%; or about 0.5% to about 1% w/w. In some embodiments, the eyedrop ophthalmic compositions comprise about 0.5% to about 1% w/w of Compound 1 or a pharmaceutically acceptable salt thereof. In some embodiments, the eyedrop ophthalmic compositions comprise about 0.5% or about 1% w/w of Compound 1 or a pharmaceutically acceptable salt thereof. In some embodiments, the eyedrop ophthalmic compositions comprise Compound 1 HCl salt.

In some embodiments, the tonicity modifier is one or more of glycerin (also known as glycerol), NaCl, and KCl. In some embodiments, the eyedrop ophthalmic compositions comprise about 0.1% to about 5% w/w of a tonicity modifier. In some embodiments, the eyedrop ophthalmic compositions comprise about 0.2% to about 2% w/w of a tonicity modifier. In some embodiments, the eyedrop ophthalmic compositions comprise about 0.5% to about 1.5% w/w of a tonicity modifier. In some embodiments, the eyedrop ophthalmic compositions comprise about 0.5% w/w of a tonicity modifier. In some embodiments, the eyedrop ophthalmic compositions comprise about 1.5% w/w of a tonicity modifier. In some embodiments, the eyedrop ophthalmic compositions comprise about 1% to about 2% w/w of glycerin. In some embodiments, the eyedrop ophthalmic compositions comprise about 1.5% w/w of glycerin.

In some embodiments, the eyedrop ophthalmic compositions comprise about 0.005% to about 0.02% w/w of a preservative. In some embodiments, the eyedrop ophthalmic compositions comprise about 0.01% w/w of a preservative. In some embodiments, the preservative is benzalkonium chloride. In some embodiments, the eyedrop ophthalmic compositions comprise about 0.005% to about 0.02% w/w of benzalkonium chloride. In some embodiments, the eyedrop ophthalmic compositions comprise about 0.01% w/w of benzalkonium chloride. In some embodiments the eyedrop ophthalmic composition does not contain a preservative.

In some embodiments, the buffer is a phosphate buffer. In some embodiments, the buffer is an about 5 mM to about 20 mM phosphate buffer. In some embodiments, the buffer is an about 10 mM phosphate buffer.

In some embodiments, the vehicle comprises water and the eyedrop ophthalmic compositions are aqueous ophthalmic compositions.

In some embodiments, provided is an aqueous ophthalmic composition comprising about 0.1% to about 2% w/w Compound 1 or a pharmaceutically acceptable salt thereof, about 1% to about 2% w/w of a tonicity modifier, about 0.005% to about 0.02% w/w of a preservative, and a buffer in water, and having a pH of about 5.5 to 7.5.

In some embodiments, provided is an aqueous ophthalmic composition comprising about 0.1% to about 2% w/w Compound 1 HCl salt, about 1% to about 2% w/w of a tonicity modifier, about 0.005% to about 0.02% w/w of a preservative, a buffer in water and having a pH of about 5.5 to 7.5.

In some embodiments, provided is an aqueous ophthalmic composition comprising about 0.1% to about 2% w/w Compound 1 HCl salt, about 1% to about 2% w/w glycerin, about 0.005% to about 0.02% w/w benzalkonium chloride, and a buffer in water, and having a pH of about 5.5 to 7.5.

In some embodiments, provided is an aqueous ophthalmic composition comprising about 0.1% to about 2% w/w Compound 1 HCl salt, about 1% to about 2% w/w glycerin, about 0.005% to about 0.02% w/w benzalkonium chloride, and a buffer in water, and having a pH of about 6.

In some embodiments, provided is an aqueous ophthalmic composition comprising about 0.1% to about 2% w/w Compound 1 HCl salt, about 1.5% w/w glycerin, about 0.005% to about 0.02% w/w benzalkonium chloride, and a buffer in water, and having a pH of about 6.

In some embodiments, provided is an aqueous ophthalmic composition comprising about 0.5% to about 1% w/w Compound 1 HCl salt, about 1% to about 2% w/w glycerin, about 0.005% to about 0.02% w/w benzalkonium chloride, and a phosphate buffer in water, and having a pH of about 5.5 to 7.5.

In some embodiments, provided is an aqueous ophthalmic composition comprising about 0.5% to 1% w/w Compound 1 HCl salt, about 1.5% w/w glycerin, about 0.01% w/w benzalkonium chloride, and about 10 mM phosphate buffer in water, and having a pH of about 6.

In some embodiments, provided is an aqueous ophthalmic composition comprising about 0.5% w/w Compound 1 HCl salt, about 1.5% w/w glycerin, about 0.01% w/w benzalkonium chloride, and about 10 mM phosphate buffer in water, and having a pH of about 6.

In some embodiments, provided is an aqueous ophthalmic composition comprising about 1% w/w Compound 1 HCl salt, about 1.5% w/w glycerin, about 0.01% w/w benzalkonium chloride, and about 10 mM phosphate buffer in water, and having a pH of about 6.

In some embodiments, the aqueous ophthalmic compositions do not comprise a stabilizer.

In some embodiments, the aqueous ophthalmic compositions further comprise a stabilizer.

Also provided are methods of using and preparing the compositions described herein.

These and other embodiments are described in more details in the text that follows.

### BRIEF DESCRIPTION OF THE DRAWINGS

FIG. 1 shows change in mean hyperemia scores, pre- to post-conjunctival allergen challenge in the mouse experiment described in Example 6.

FIG. 2A shows the baseline imaging of conjunctiva using in vivo confocal microscopy to assess the micro vasculature, and to score the inflammation on a scale from 0 (no white blood cells) to 4 (visible inflammation of cells) described in Example 7. FIG. 2B shows the imaging of conjunctiva post allergen challenge (CAC) which was 8 hours later after treatment. In FIGS. 2A and 2B, from left to right: Vehicle (N=3), Patanol® (N=8), Composition B, 1% Compound 1 (N=7), and Composition A, 0.5% Compound 1 (N=5).

### DETAILED DESCRIPTION

### Definitions

Unless defined otherwise, all technical and scientific terms used herein have the same meaning as is commonly understood by one of ordinary skill in the art. As used herein, the below terms have the following meanings unless specified otherwise. Any methods, devices and materials similar or equivalent to those described herein can be used in the practice of the compositions and methods described herein. The following definitions are provided to facilitate understanding of certain terms used frequently herein and are not meant to limit the scope of the present disclosure. All references referred to herein are incorporated by reference in their entirety.

Headings used in this application are for reference purposes only and do not in any way limit the present disclosure.

The term “comprise” and variations thereof, such as, “comprises” and “comprising” are to be construed in an open, inclusive sense, that is, as “including, but not limited to.” “Consisting essentially of” or its grammatic variants when used to define compositions and methods, shall mean excluding other elements of any essential significance to the compositions and methods for the intended use, but not excluding elements that do not materially affect the characteristic(s) of the compositions or methods. “Consisting of” or its grammatic variants shall mean excluding elements not specifically recited. Embodiments defined by each of these transition terms are within the scope of this disclosure. For example, when a composition is described as comprising ingredients A, B and C, a composition consisting essentially of A, B and C, and a composition consisting of A, B and C are independently within the scope of this disclosure.

It is noted here that as used in this specification and the appended claims, the singular forms “a” “an” and “the” and the like include plural referents unless the context clearly dictates otherwise. For example, the term “a pharmaceutically acceptable vehicle” includes reference to one and more than one pharmaceutically acceptable vehicles.

The term “about” means within ±20%, 15%, 10%, 9%, 8%, 7%, 6%, 5%, 4%, 3%, 2%, 1%, 0.5%, or 0.05% of a given value or range. In one embodiment, about means ±5% of a given value or range. In another embodiment, “about” means ±4% of a given value or range. In another embodiment, “about” means ±3% of a given value or range. In another embodiment, “about” means ±2% of a given value or range. In another embodiment, “about” means ±1% of a given value or range. In another embodiment, “about” means ±0.5% of a given value or range. In another embodiment, “about” means ±0.05% of a given value or range. The term “about x” includes the value “x.”

The term “administration” refers to introducing an agent to a patient. A therapeutic amount can be administered, which can be determined by the treating physician or the like. The related terms and phrases “administering” and “administration of,” when used in connection with a compound or composition (and grammatical equivalents) refer both to direct administration, which may be administration to a patient by a medical professional or by self-administration by the patient, and/or to indirect administration, which may be the act of prescribing a drug. Administration entails delivery to the patient of the drug.

The term “dose” or “dosage” refers to the total amount of active ingredient (e.g., Compound 1 or a pharmaceutically acceptable salt thereof) administered to a patient in a single administration. The terms “dose” and “dosage” are used interchangeably herein.

“Therapeutically effective amount” or “therapeutic amount” refers to an amount of a drug or an agent that when administered to a patient suffering from a condition or disease, will have the intended therapeutic effect, e.g., reducing or curing the disease, alleviation, amelioration, palliation or elimination of one or more symptoms or manifestations of the condition or disease in the patient. The therapeutic effect does not necessarily occur by administration of one dose, and may occur after administration of a series of doses over a period of time, such as one day, two days, three days, four days, five days, one week, two weeks, three weeks, one month, etc. or as long as needed and appropriate.

The term “pharmaceutically acceptable” refers to generally safe and non-toxic for in vivo, preferably human, administration.

The term “patient” refers to a mammal, such as a human, bovine, rat, mouse, dog, cat, monkey, ape, goat, sheep, cow, horse, or deer. A patient as described herein can be a human. In some embodiments, the patient is an adult. In some embodiments, the patient is a child or juvenile.

“Treatment,” “treating,” and “treat” are defined as acting upon a disease, disorder, or condition with an agent to reduce or ameliorate the harmful or any other undesired effects of the disease, disorder, or condition and/or its symptoms. Treatment, as used herein, covers the treatment of a human patient, and includes: (a) reducing the risk of occurrence of the condition or disease in a patient determined to be predisposed to the condition or disease but not yet diagnosed as having the condition or disease, (b) impeding the development of the condition or disease, and/or (c) relieving the condition or disease, i.e., causing regression of the condition or disease and/or relieving one or more symptoms of the condition or disease. For purposes of treatment of an ophthalmic disease or condition, beneficial or desired clinical results include, but are not limited to, reduction or elimination of an allergic reaction and/or inflammation, reduction or elimination of one or more symptoms of the ophthalmic disease, such as reduction or elimination of ocular itching, and/or reduction or elimination of conjunctival redness, reduction of ocular discomfort, reduction of corneal or conjunctival staining, and the like, including any other symptom or combination of symptoms provided herein.

As used herein, “% w/w” refers to the weight of a component based on the total weight of a composition comprising the component. For instance, if component 1 is present in an amount of 50 mg in a 100 mg composition, component 1 is present in an amount of 50% w/w. It is to be understood that “% w/w” refers to the percent weight of an agent or excipient relative to the total weight of the composition as described herein unless explicitly stated otherwise. Percent weights described herein do not include the weight of a container unless explicitly stated as such.

### Methods of Treatment

Provided herein are methods of using a Syk inhibitor in the treatment of ophthalmic diseases. In some embodiments, provided is a method of treating an ophthalmic disease or condition comprising administering a therapeutically effective amount of a Syk inhibitor topically to an eye of a patient in need thereof. In some embodiments, provided herein is use of a Syk inhibitor in the treatment of an ophthalmic disease or condition. In some embodiments, provided herein is use of a Syk inhibitor in the preparation of a medicament for the treatment of an ophthalmic disease or condition.

In some embodiments, the Syk inhibitor is administered in an ophthalmic composition once a day, twice a day, three times a day, or four times a day. In some embodiments, the Syk inhibitor may be administered by a sustained release drug delivery mechanism. In some embodiments, the method comprises administering about 0.001 mg to about 10 mg of the Syk inhibitor topically to an eye of a patient in need thereof once, twice or three times a day. In some embodiments, the method comprises administering about 0.001 mg, about 0.005 mg, about 0.01 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 3 mg, about 4 mg, about 5 mg, about 6 mg, about 7 mg, or about 8 mg or any range between any two of the values (end point inclusive) of the Syk inhibitor to an eye of a patient in need thereof once, twice or three times a day. In some embodiments, the method comprises administering one or two drops of an ophthalmic composition comprising the Syk inhibitor topically to an eye of a patient in need thereof once a day, twice a day, three times a day, or four times a day.

In some embodiments, provided herein is a method for treating an ophthalmic disease or condition comprising administering to a patient in need thereof a therapeutically effective amount of Compound 1 or a pharmaceutically acceptable salt thereof.

In some embodiments, provided herein is use of Compound 1 or a pharmaceutically acceptable salt thereof in the treatment of an ophthalmic disease or condition. In some embodiments, provided herein is use of Compound 1 or a pharmaceutically acceptable salt thereof in the preparation of a medicament for the treatment of an ophthalmic disease or condition.

Compound 1 has the chemical name: 2-((1R,2S)-2-aminocyclohexylamino)-4-(3-(pyrimidin-2-yl)phenylamino)pyrimidine-5-carboxamide, and is of the formula:

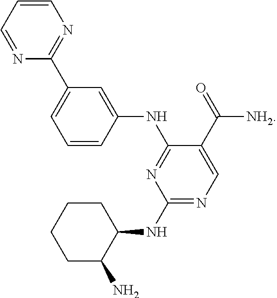 [SMILES: NC(=O)c1cnc(N[C@@H]2CCCC[C@@H]2N)nc1Nc1cccc(-c2ncccn2)c1]

It is described in U.S. Pat. No. 8,318,755, which is hereby incorporated by reference in its entirety.

Compound 1 or a pharmaceutically acceptable salt thereof is also referred to herein as the active ingredient or API. Pharmaceutically acceptable salts of Compound 1 include acid addition salt whose counter-ions are non-toxic to the patient in pharmaceutical doses of the salts, such as acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. In some embodiments, the pharmaceutically acceptable salt of Compound 1 is one or more of a formate, oxalate, maleate, citrate, phosphate or hydrochloride salt of Compound 1. In some embodiments, the pharmaceutically acceptable salt of Compound 1 is one or more of a formate, maleate, citrate, phosphate or hydrochloride salt of Compound 1. In some embodiments, pharmaceutically acceptable salt of Compound 1 is a hydrochloride salt of Compound 1, which is also referred to as Compound 1 HCl salt or Compound 1 hydrochloride. In some embodiments, the ophthalmic compositions, such as aqueous ophthalmic compositions, comprise a cation of Compound 1, and an anion of an acid described herein.

In some embodiments, the ophthalmic disease or condition is allergic and/or inflammatory, including one or more of allergic conjunctivitis (also called ocular allergy or eye allergy, including acute allergic conjunctivitis, chronic allergic conjunctivitis, temporary allergic conjunctivitis, persistent allergic conjunctivitis, seasonal allergic conjunctivitis or perennial allergic conjunctivitis), rhinoconjunctivis, dry eye, keratoconjunctivitis, eye inflammation, inflammation of the ocular surface or eyelids (e.g., dry eye, blepharitis, ptyergium, peripheral corneal infiltrate, post corneal transplant, pingueculitis, episcleritis, scleritis, keratitis, fungal keratitis, dermatitis of the eyelids, bacterial and viral conjunctivitis, atopic keratoconjunctivitis (AKC), neurotrophic keratitis, and GVHD-graft versus host disease), other ocular surface inflammation, irritation, and/or hyperemia, and/or anterior chamber of the eye (e.g., anterior uveitis, post-operative inflammation, traumatic and post-surgical iritis), vernal keratoconjunctivitis (VKC), iritis, uveitis, pingueculum, pterygium, contact lens induced dry eye, steroid-responsive inflammation of the palpebral and bulbar conjunctiva, cornea, and anterior segment of the globe, posterior uveitis, retina diseases such as macular edema associated with cystoid macular edema, diabetic macular edema, branch retinal vein occlusion (BRVO), central retinal vein occlusion (CRVO), eye redness, swelling eye, eyelid swelling, and itchy eye, and ocular conditions for which a corticosteroid is indicated. In some embodiments, the ophthalmic disease or condition is acute or chronic allergic conjunctivitis, which may be seasonal, perennial, temporary, or persistent allergic conjunctivitis.

In some embodiments, the ophthalmic disease or condition is one or more of allergic conjunctivitis (such as acute allergic conjunctivitis, chronic allergic conjunctivitis, temporary allergic conjunctivitis, persistent allergic conjunctivitis, seasonal allergic conjunctivitis or perennial allergic conjunctivitis), rhinoconjunctivis, dry eye, keratoconjunctivitis, blepharitis, dermatitis of the eyelids, blepharoconjunctivitis, viral conjunctivitis, bacterial conjunctivitis, other infection caused by virus, bacteria, or fungus, eye inflammation, irritation and/or hyperemia, inflammation of the ocular surface, eyelids, or anterior or posterior chamber of the eye, atopic keratoconjunctivitis (AKC), vernal keratoconjunctivitis (VKC), neurotrophic keratitis, GVHD-graft versus host disease, traumatic or post-surgical iritis, scleritis, episleritis, keratitis, uveitis, pingueculum, pterygium, contact lens induced dry eye, posterior uveitis, retina diseases such as macular edema associated with cystoid macular edema, diabetic macular edema, branch retinal vein occlusion (BRVO), central retinal vein occlusion (CRVO), eye redness, eyelid swelling, eyelid congestion, swelling eye, and itchy eye, steroid-responsive inflammation of the palpebral and bulbar conjunctiva, cornea, and anterior segment of the globe, and ocular conditions for which a corticosteroid is indicated. In some embodiments, the ophthalmic disease or condition is acute or chronic allergic conjunctivitis, which may be seasonal, perennial, temporary, or persistent.

In some embodiments, the ophthalmic disease or condition is an anterior segment inflammatory disease. In some embodiments, the ophthalmic disease or condition is an ocular surface inflammatory disease, such as dry eye, blepharitis, ptyergium, peripheral corneal infiltrate, post corneal transplant, pingueculitis, episcleritis, scleritis, atopic keratoconjunctivitis, vernal keratoconjunctivitis, fungal keratitis (via effect of TLR signaling), bacterial or viral conjunctivitis (treating the inflammatory component—not necessarily as an anti-infective), steroid-responsive inflammation of the palpebral and bulbar conjunctiva, cornea, and anterior segment of the globe, and ocular conditions for which a corticosteroid is indicated. In some embodiments, the ophthalmic disease or condition is an anterior chamber inflammatory disease, such as anterior uveitis, post-operative inflammation, or traumatic or post-surgical iritis.

In some embodiments, the ophthalmic disease or condition is acute allergic conjunctivitis, which may be seasonal, perennial, temporary, or persistent. In some embodiments, the ophthalmic disease or condition is chronic allergic conjunctivitis, which may be seasonal, perennial, temporary, or persistent.

In some embodiments, the method treats a symptom of the ophthalmic disease or condition, including redness, inflammation, irritation, swelling of the eyelids, eyelid congestion, chemosis (swelling of the conjunctiva), watery eye, itching, burning, foreign body sensation, and/or other discomfort, dryness, grittiness, burning, keratitis, conjunctival redness, conjunctival staining, corneal staining, reduced tearing, reduced tear film break up time, reduced quality of life, reduced visual function, or a combination of thereof. Symptoms of dry eye include, but are not limited to stinging or burning of the eye; a sandy or gritty feeling as if something is in the eye; episodes of excess tears following very dry eye periods; a stringy discharge from the eye; pain and redness of the eye; episodes of blurred vision; heavy eyelids; inability to cry when emotionally stressed; uncomfortable contact lenses; decreased tolerance of reading, working on the computer, or any activity that requires sustained visual attention; and eye fatigue.

In some embodiments, the method treats a symptom of acute or chronic allergic conjunctivitis, including ocular itching, redness, such as conjunctival redness, episcleral and ciliary redness, inflammation, swelling of the eyelids, chemosis, watery eye and sensitivity to light.

In some embodiments, the method further treats one or more of other allergic symptoms including nasal allergic symptoms, such as nasal congestion, rhinorrhea, and nasal pruritis, ear or palate pruritis, and allergic headaches.

In some embodiments, the method treats a symptom of dry eye including stinging and/or burning sensation, gritty sensation, episodes of excess tears, stringy discharge, pain, redness, blurred vision, heavy eyelid, inability to cry, discomfort, for example, when wearing contact lenses, decreased tolerance of visual attention, and eye fatigue.

In some embodiments, the allergic conjunctivitis is caused by a perennial allergen (e.g., cat dander, dog dander, dust mites, and/or cockroaches) and/or a seasonal allergen (e.g., pollens of trees, grasses, and/or ragweed) or pollutants. In some embodiments, the dry eye is caused by environmental factors, nutrition, inflammatory disease, systemic disease, hydration level, genetic factors, neurotrophic condition or disease, neurological condition, or other dysfunction of the tear film (tear production, mucin production, lipid production), or other dysregulation of the ocular surface.

In some embodiments, the ophthalmic disease or condition is allergic and/or inflammatory, including signs and/or symptoms of one or more of allergic conjunctivitis (including acute allergic conjunctivitis, chronic allergic conjunctivitis, seasonal allergic conjunctivitis or perennial allergic conjunctivitis), rhinoconjunctivis, dry eye, keratoconjunctivitis, blepharitis, dermatitis of the eyelids, blepharoconjunctivitis, ptyergium, post corneal transplant, pingueculitis, episcleritis, scleritis, keratitis, peripheral corneal infiltrate, fungal keratitis, bacterial and viral conjunctivitis, post-operative inflammation, eye inflammation, inflammation of the ocular surface or eyelids, anterior chamber or posterior chamber of the eye, atopic keratoconjunctivitis (AKC), vernal keratoconjunctivitis (VKC), giant papillary conjunctivitis (GPC), neurotrophic keratitis, GVHD-graft versus host disease, traumatic or post-surgical iritis, uveitis, pingueculum, pterygium, contact lens induced dry eye, other ocular surface inflammation, irritation, and/or hyperemia, posterior uveitis, retina diseases, diabetic macular edema, branch retinal vein occlusion (BRVO), central retinal vein occlusion (CRVO), eye redness, eyelid swelling, eyelid congestion, swelling eye, and itchy eye, steroid-responsive inflammation of the palpebral and bulbar conjunctiva, cornea, and anterior segment of the globe, and ocular conditions for which a corticosteroid is indicated.

In some embodiments, the ophthalmic disease or condition is one or more of dry eye, blepharitis, ptyergium, peripheral corneal infiltrate, post corneal transplant, pingueculitis, episcleritis/scleritis, atopic keratoconjunctivitis, fungal keratitis, allergy, AKC, VKC, GPC, bacterial or viral conjunctivitis, anterior uveitis, traumatic or post-surgical iritis, eyelid swelling, eye redness, irritation, ocular surface inflammation, or post-operative inflammation.

In some embodiments, one or more of redness, inflammation, swelling, discomfort, watery eye and itching of the eye, keratitis, corneal staining, conjunctival staining, or markers of inflammation of the eye is reduced or eliminated.

In some embodiments, the ophthalmic disease is one or more of dry eye, allergic conjunctivitis, keratoconjunctivitis (sicca), keratitis, blepharitis, ptyergium, peripheral corneal infiltrate, post corneal transplant, pingueculitis, episcleritis/scleritis, atopic keratoconjunctivitis, fungal keratitis, bacterial and viral conjunctivitis, anterior uveitis, or post-operative inflammation, and signs and/or symptoms thereof.

In addition, the clinical model of allergy, the conjunctival allergen challenge (CAC), which is accepted by FDA, may be a standard for screening and development of new products. The CAC may be useful for screening of novel anti-inflammatory agents to identify potential use for conditions and diseases other than allergy specifically. The CAC can be used for dose ranging, proof of concept, and identification of specific anti-inflammatory effects.

Compound 1 or a pharmaceutically acceptable salt thereof may be administered in a suitable composition, such as in form of a solution, suspension, emulsion, ointment, gel, spray, depots, or a sustained release formulation implant or depot, etc., either locally as eyedrop or ocular injection, implant, or insert, or systemically. When administered locally, Compound 1 or a pharmaceutically acceptable salt thereof may be administered to one or both eyes. It may be administered to a naked eye, an eye with a contact lens, or within or on a contact lens or contact lens packing solution. Compound 1 or a pharmaceutically acceptable salt thereof may be administered once a day, twice a day, three times a day, four times a day or more frequently at appropriate intervals throughout the day, or as needed. In some embodiments, Compound 1 or a pharmaceutically acceptable salt thereof is administered once a day. In some embodiments, Compound 1 or a pharmaceutically acceptable salt thereof is administered twice a day. In some embodiments, Compound 1 is administered in an ophthalmic composition comprising Compound 1 or a pharmaceutically acceptable salt thereof and a pharmaceutically acceptable vehicle. Pharmaceutically acceptable vehicles include carriers, diluents or excipients suitable for ophthalmic use, for example, generally speaking acceptable vehicles do not produce undesirable irritation itself, and do not trigger a secretion of tears that will entrain the active ingredient. In some embodiments, Compound 1 or a pharmaceutically acceptable salt thereof is administered in a composition described herein.

In some embodiments, Compound 1 or a pharmaceutically acceptable salt thereof may be administered by a sustained release drug delivery system that releases Compound 1 over time. For example, a sustained release drug delivery system may deliver about 0.001 mg to about 10 mg of Compound 1 to an eye of a patient in need thereof. In some embodiments, the method comprises administering about 0.001 mg, about 0.005 mg, about 0.01 mg, about 0.05 mg, about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 3 mg, about 4 mg, about 5 mg, about 6 mg, about 7 mg, or about 8 mg or any range between any two of the values (end point inclusive) of Compound 1 to an eye of a patient. The sustained drug delivery system may deliver Compound 1 over a period of about 1 minute, about 2 minutes, about 5 minutes, about 10 minutes, about 15 minutes, about 20 minutes, about 30 minutes, about 45 minutes, about 1 hour, about 2 hours, about 3 hours, about 1 day, about 1 week, about 1 month, or about 1 year. In some embodiments, Compound 1 may be administered for a time period as determined by a medical practitioner.

In some embodiments, the method further comprises administering another agent such as an anti-histamine, vasoconstrictor, antibiotic, anti-inflammatory, immunosuppressant, an agent for relieving dry eye or discomfort or signs, anti-vascular agent, anti-fibrotic, anti-angiogenic, wound healing agent, etc., either as a fixed combination or dosed concomitantly or adjunctively.

### Pharmaceutical Compositions

Provided herein are also pharmaceutical compositions, in particular eyedrop ophthalmic compositions, comprising Compound 1, or a pharmaceutically acceptable salt thereof, and a pharmaceutically acceptable liquid vehicle, suitable for treating ophthalmic diseases or conditions, such as those described herein. The eyedrop liquid vehicle may be aqueous or non-aqueous in nature. In some embodiments, the vehicle comprises water and the eyedrop ophthalmic composition is an aqueous ophthalmic composition.

In some embodiments, the compositions provided herein are stable clear liquids suitable for use as eye drops with minimum number of excipients that deliver an efficacious amount of the active ingredient and produce minimum or no side effects or discomfort to the eye.

In some embodiments, the ophthalmic compositions, such as aqueous ophthalmic compositions, comprise a pharmaceutically acceptable salt of Compound 1. In some embodiments, the pharmaceutically acceptable salt of Compound 1 is one or more of a formate, oxalate, maleate, citrate, phosphate or hydrochloride salt of Compound 1. In some embodiments, pharmaceutically acceptable salt of Compound 1 is Compound 1 HCl salt.

In some embodiments, the ophthalmic compositions, such as aqueous ophthalmic compositions, comprise Compound 1 and/or a cation of Compound 1, and an anion of an acid. In some embodiments, the anion of the acid is one or more of a formate anion, oxalate anion, maleate anion, citrate anion, phosphate anion or chloride anion (Cl⁻).

In some embodiments, provided are eyedrop ophthalmic compositions comprising Compound 1 or a pharmaceutically acceptable salt thereof, a liquid vehicle, and an excipient, which can comprise one or more of a buffer, a tonicity modifier, a stabilizer, a solubilizer, a preservative, a surfactant, a demulcent, a viscosifier, a chelating agent, an anti-oxidant agent, and a penetration enhancing agent.

In some embodiments, provided are aqueous ophthalmic compositions comprising Compound 1 or a pharmaceutically acceptable salt thereof, water, and an excipient, which can comprise one or more of a buffer, a tonicity modifier, a stabilizer, a preservative, a surfactant, a demulcent, a viscosifier, a chelating agent, an anti-oxidant agent, and a penetration enhancing agent.

In some embodiments, provided are aqueous ophthalmic compositions prepared by a method comprising mixing Compound 1 or a pharmaceutically acceptable salt thereof, water, and an excipient, which can comprise one or more of a buffer, a tonicity modifier, a stabilizer, a preservative, a surfactant, a demulcent, a viscosifier, a chelating agent, an anti-oxidant agent, and a penetration enhancing agent. In some embodiments, the method further comprises adjusting the pH of the composition.

In some embodiments, the aqueous ophthalmic compositions are clear solutions. In other embodiments, the ophthalmic composition may be non-aqueous liquid.

In some embodiments, the buffer is selected from borate buffers, phosphate buffers, carbonate buffers, and acetate buffers, or a combination thereof.

In some embodiments, the tonicity modifier is one or more of glycerin (also known as glycerol), sodium chloride (NaCl), potassium chloride (KCl), dextrose, sucrose, mannitol, sorbitol, polyethylene glycol (PEG), PEG 3350, magnesium citrate, lactulose, and colloidal osmotics such as pentastarch, hetastarch, gelatin polypetides, dextran, albumin, alginate, and crystalline cellulose derivatives, or a combination thereof. In some embodiments, the tonicity modifier is one or more of glycerin, NaCl, and KCl.

In some embodiments, the preservative is selected from chlorobutanol, sodium dehydroacetate, benzalkonium chloride (BAC), cetyl pyridinium chloride, phenethyl alcohol, parahydroxybenzoic acid esters (such as methyl, ethyl, propyl or butyl ester), benzethonium chloride, sodium perborate, sepazonium, iodine, polyquad, sodium chlorite, and hypochlorous acids, or a combination thereof. In some embodiments, the preservative is benzalkonium chloride.

In some embodiments, the viscosity-increasing agent is selected from methylcellulose, hydroxyethylcellulose, carboxymethylcellulose, hydroxypropylmethylcellulose, polyvinyl alcohol, carboxymethylcellulose, chondroitin sulfate, and salts thereof, or a combination thereof.

Examples of chelating agents include sodium edetate and citric acid.

Examples of stabilizers include sodium edetate, sodium hydrogen sulfite and stabilizing agents as defined in U.S. Patent Application Publication 2007/0265234 which is hereby incorporated by reference in its entirety.

In some embodiments, the stabilizer is one or more of polysorbate (such as polysorbate 20, polysorbate 40, polysorbate 60, or polysorbate 80), PEG-35 castor oil (such as PEG-30 castor oil, PEG-33 castor oil, PEG-35 castor oil, PEG-36 castor oil or PEG-40 castor oil), and polyvinylpyrrolidone (also known as povidone or PVP, such as PVP K20, PVP K25, PVP K29/32, PVP K32, PVP K35 and PVP K40).

Examples of solubilizers include, but are not limited to, polyoxyethylene hydrogenated castor oil, polyethylene glycol, polysorbate 80, polyoxyethylene monostearate, and semi-fluorinated alkanes.

In some embodiments, the ophthalmic compositions, such as aqueous ophthalmic compositions do not comprise a stabilizer or a solubilizer.

It is contemplated that an eyedrop may range from 25 μL (microliters) to 50 μL. In some embodiments, eye drops are administered to the patient in 1 to 2 drops to each eye. As a nonlimiting example, a single drop of a 5 to 10 mg/mL composition of Compound 1 may contain 0.125 mg to 0.5 mg of Compound 1. Thus, in a nonlimiting example, a patient receives as much as 2 drops (1 mg total) in each eye (2 mg between both eyes) twice a day, and as much as 4 mg is administered to the patient total per day.

The ophthalmic compositions, such as aqueous ophthalmic compositions, described herein can comprise or deliver to the eye Compound 1 or a pharmaceutically acceptable salt thereof in an amount (% w/w) of about 0.001% to about 10%; about 0.01% to about 10%; about 0.05% to about 10%; about 0.1% to about 1%; about 0.1% to about 2%; about 0.1% to about 5%; about 0.1% to about 10%; about 0.2% to about 7%; about 0.3% to about 5%; about 0.4% to about 2%; or about 0.5% to about 1% w/w. In some embodiments, the ophthalmic compositions, such as aqueous ophthalmic compositions, comprise Compound 1 or a pharmaceutically acceptable salt thereof in an amount (% w/w) of about 0.001% to about 7%; about 0.001% to about 5%; about 0.001% to about 2%; or about 0.001% to about 1% w/w. In some embodiments, the ophthalmic compositions, such as aqueous ophthalmic compositions, comprise Compound 1 or a pharmaceutically acceptable salt thereof in an amount (% w/w) of about 0.01% to about 7%; about 0.01% to about 5%; about 0.01% to about 2%; or about 0.01% to about 1% w/w. For example, the ophthalmic compositions, such as aqueous ophthalmic compositions, can comprise (% w/w) about 0.001%, about 0.005%, about 0.01%, about 0.05%, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.1%, about 2.2%, about 2.3%, about 2.4%, about 2.5%, about 2.6%, about 2.7%, about 2.8%, about 2.9%, or about 3% w/w of Compound 1 or a pharmaceutically acceptable salt thereof, or any range between any two of the numbers, end points inclusive. In some embodiments, ophthalmic compositions, such as aqueous ophthalmic compositions, comprise about 0.5% to about 1% w/w of Compound 1 or a pharmaceutically acceptable salt thereof. In some embodiments, the ophthalmic compositions, such as aqueous ophthalmic compositions, comprise about 0.5% or about 1% w/w of Compound 1 or a pharmaceutically acceptable salt thereof.

In some embodiments, the ophthalmic compositions, such as aqueous ophthalmic compositions have a pH of from about 5.5 to about 8. In some embodiments, the ophthalmic compositions, such as aqueous ophthalmic compositions have a pH of from about 6 to about 7.5. In some embodiments, the ophthalmic compositions, such as aqueous ophthalmic compositions have a pH of from about 6.5 to about 7.3. In some embodiments, the ophthalmic compositions, such as aqueous ophthalmic compositions have a pH of from about 6.8 to about 7.2. In some embodiments, the ophthalmic compositions, such as aqueous ophthalmic compositions have a pH of from about 7. In some embodiments, the ophthalmic compositions, such as aqueous ophthalmic compositions have a pH of from about 5.5 to about 7. In some embodiments, the ophthalmic compositions, such as aqueous ophthalmic compositions have a pH of from about 5.7 to about 6.5. In some embodiments, the ophthalmic compositions, such as aqueous ophthalmic compositions have a pH of from about 5.9 to about 6.3. In some embodiments, the ophthalmic compositions, such as aqueous ophthalmic compositions have a pH of from about 6 to about 6.2. In some embodiments, the ophthalmic compositions, such as aqueous ophthalmic compositions have a pH of about 6.

In some embodiments, the pH of the compositions is adjusted by one or more of sodium hydroxide, potassium hydroxide, sodium carbonate, citric acid, phosphoric acid, acetic acid, and hydrochloric acid.

In some embodiments, the aqueous ophthalmic compositions comprise Compound 1 or a pharmaceutically acceptable salt thereof, a buffer to adjust the pH of the composition, and water. In some embodiments, the buffer is selected from borate buffers, phosphate buffers, citrate buffers, carbonate buffers, and acetate buffers. In some embodiments, the concentration of buffer in the ophthalmic compositions is from about 1 mM to about 150 mM or more, depending on the particular buffer chosen. In some embodiments, the concentration of buffer is less than 100 mM, such as from about 1 mM to about 25 mM, or from about 1 mM to about 20 mM.

In some embodiments, the buffer is a citrate buffer or a phosphate buffer. In some embodiments, the buffer is an about 5 mM to about 20 mM citrate buffer. In some embodiments, the buffer is an about 5 mM to about 20 mM phosphate buffer. In some embodiments, the buffer is an about 10 mM citrate buffer. In some embodiments, the buffer is an about 10 mM phosphate buffer.

In some embodiments, the aqueous ophthalmic compositions have an osmolality of from about 200 to about 350 mOsm/kg. In some embodiments, the aqueous ophthalmic compositions have an osmolality of from about 230 to about 310 mOsm/kg.

In some embodiments, the aqueous ophthalmic compositions comprise Compound 1 or a pharmaceutically acceptable salt thereof, a tonicity modifier to adjust the osmolality of the composition, and water.

In some embodiments, the aqueous ophthalmic compositions comprise about 0.1% to about 5% w/w of a tonicity modifier. In some embodiments, the aqueous ophthalmic compositions comprise about 0.2% to about 2% w/w of a tonicity modifier. In some embodiments, the aqueous ophthalmic compositions comprise about 0.5% to about 1.5% w/w of a tonicity modifier. In some embodiments, the aqueous ophthalmic compositions comprise about 0.5% w/w of a tonicity modifier. In some embodiments, the aqueous ophthalmic compositions comprise about 1.5% w/w of a tonicity modifier. In some embodiments, the aqueous ophthalmic compositions comprise about 0.2% to about 1% w/w of NaCl. In some embodiments, the aqueous ophthalmic compositions comprise about 0.5% w/w of NaCl. In some embodiments, the aqueous ophthalmic compositions comprise about 1% to about 2% w/w of glycerin. In some embodiments, the aqueous ophthalmic compositions comprise about 1.5% w/w of glycerin.

In some embodiments, the aqueous ophthalmic compositions comprise Compound 1 or a pharmaceutically acceptable salt thereof, a buffer, a tonicity modifier, and water.

In some embodiments, the aqueous ophthalmic compositions further comprise a preservative to prevent decomposition or microbial growth. In some embodiments, the aqueous ophthalmic compositions comprise about 0.005% to about 0.02% w/w of a preservative. In some embodiments, the aqueous ophthalmic compositions comprise about 0.01% w/w of a preservative.

In some embodiments, the aqueous ophthalmic compositions comprise about 0.005% to about 0.02% w/w of benzalkonium chloride. In some embodiments, the aqueous ophthalmic compositions comprise about 0.01% w/w of benzalkonium chloride.

In some embodiments, the aqueous ophthalmic compositions further comprise a stabilizer to prevent physical changes, such as precipitation.

In some embodiments, the aqueous ophthalmic compositions comprise about 0% to about 5% w/w of a stabilizer. In some embodiments, the aqueous ophthalmic compositions comprise about 1% to about 3% w/w of a stabilizer. In some embodiments, the aqueous ophthalmic compositions comprise about 2% w/w of a stabilizer.

In some embodiments, the stabilizer is one or more of polysorbate 80 (PS80, available under brand names Montanox™ 80, Alkest® TW 80 and Tween® 80), PEG-35 castor oil (also known as polyoxyl 35 hydrogenated castor oil, polyoxyl-35 castor oil, macrogolglycerol ricinoleate, and available under brand names Kolliphor® EL, Kolliphor® ELP and Cremophor® EL), and povidone K29/32 (PVP K29/32, available under the trade name Plasdone™ K-29/32). In some embodiments, the aqueous ophthalmic compositions comprise about 0% to about 5% w/w of polysorbate 80, PEG-35 castor oil or PVP K29/32.

In some embodiments, the aqueous ophthalmic compositions comprise about 1% to about 3% w/w of polysorbate 80, PEG-35 castor oil or PVP K29/32. In some embodiments, the aqueous ophthalmic compositions comprise about 2% w/w of polysorbate 80, PEG-35 castor oil or PVP K29/32.

In some embodiments, the aqueous ophthalmic compositions do not comprise a stabilizer and yet are surprisingly stable.

In some embodiments, the aqueous ophthalmic compositions comprise a solubilizer. Examples of solubilizers include, but are not limited to, polyoxyethylene hydrogenated castor oil, polyethylene glycol, polysorbate 80, polyoxyethylene monostearate. In some embodiments, the aqueous ophthalmic compositions do not comprise a solubilizer.

In some embodiments, the aqueous ophthalmic compositions comprise a viscosity-increasing agent.

In some embodiments, the aqueous ophthalmic compositions comprise a chelating agent. In some embodiments, the chelating agent is sodium edetate or citric acid.

In some embodiments, provided is an ophthalmic composition comprising about 0.1% w/w to about 2% w/w of Compound 1 or a pharmaceutically acceptable salt thereof, a tonicity modifier, a buffer, and water.

In some embodiments, provided is an ophthalmic composition comprising about 0.5% w/w to about 1% w/w of Compound 1 or a pharmaceutically acceptable salt thereof, a tonicity modifier, a buffer, and water.

In some embodiments, provided is an aqueous ophthalmic composition comprising about 0.001% to about 2% w/w Compound 1 or a pharmaceutically acceptable salt thereof, about 1% to about 2% w/w of a tonicity modifier, about 0.005% to about 0.02% w/w of a preservative, and a buffer in water, and having a pH of about 5.5 to 7.5.

In some embodiments, provided is an aqueous ophthalmic composition comprising about 0.01% to about 2% w/w Compound 1 or a pharmaceutically acceptable salt thereof, about 1% to about 2% w/w of a tonicity modifier, about 0.005% to about 0.02% w/w of a preservative, and a buffer in water, and having a pH of about 5.5 to 7.5.

In some embodiments, provided is an aqueous ophthalmic composition comprising about 0.1% to about 2% w/w Compound 1 or a pharmaceutically acceptable salt thereof, about 1% to about 2% w/w of a tonicity modifier, about 0.005% to about 0.02% w/w of a preservative, and a buffer in water, and having a pH of about 5.5 to 7.5.

In some embodiments, provided is an aqueous ophthalmic composition comprising about 0.001% to about 2% w/w Compound 1 HCl salt, about 1% to about 2% w/w of a tonicity modifier, about 0.005% to about 0.02% w/w of a preservative, a buffer in water and having a pH of about 5.5 to 7.5.

In some embodiments, provided is an aqueous ophthalmic composition comprising about 0.01% to about 2% w/w Compound 1 HCl salt, about 1% to about 2% w/w of a tonicity modifier, about 0.005% to about 0.02% w/w of a preservative, a buffer in water and having a pH of about 5.5 to 7.5.

In some embodiments, provided is an aqueous ophthalmic composition comprising about 0.1% to about 2% w/w Compound 1 HCl salt, about 1% to about 2% w/w of a tonicity modifier, about 0.005% to about 0.02% w/w of a preservative, a buffer in water and having a pH of about 5.5 to 7.5.

In some embodiments, provided is an aqueous ophthalmic composition comprising about 0.1% to about 2% w/w Compound 1 HCl salt, about 1% to about 2% w/w glycerin, about 0.005% to about 0.02% w/w benzalkonium chloride, and a buffer in water, and having a pH of about 5.5 to 7.5.

In some embodiments, provided is an aqueous ophthalmic composition comprising about 0.1% to about 2% w/w Compound 1 HCl salt, about 1% to about 2% w/w glycerin, about 0.005% to about 0.02% w/w benzalkonium chloride, and a buffer in water, and having a pH of about 6.

In some embodiments, provided is an aqueous ophthalmic composition comprising about 0.1% to about 2% w/w Compound 1 HCl salt, about 1.5% w/w glycerin, about 0.005% to about 0.02% w/w benzalkonium chloride, and a buffer in water, and having a pH of about 6.

In some embodiments, provided is an aqueous ophthalmic composition comprising about 0.5% to about 1% w/w Compound 1 HCl salt, about 1% to about 2% w/w glycerin, about 0.005% to about 0.02% w/w benzalkonium chloride, and a phosphate buffer in water, and having a pH of about 5.5 to 7.5.

In some embodiments, provided is an aqueous ophthalmic composition comprising about 0.5% to 1% w/w Compound 1 HCl salt, about 1.5% w/w glycerin, about 0.01% w/w benzalkonium chloride, and about 10 mM phosphate buffer in water, and having a pH of about 6.

In some embodiments, provided is an aqueous ophthalmic composition comprising about 0.5% w/w Compound 1 HCl salt, about 1.5% w/w glycerin, about 0.01% w/w benzalkonium chloride, and about 10 mM phosphate buffer in water, and having a pH of about 6.

In some embodiments, provided is an aqueous ophthalmic composition comprising about 1% w/w Compound 1 HCl salt, about 1.5% w/w glycerin, about 0.01% w/w benzalkonium chloride, and about 10 mM phosphate buffer in water, and having a pH of about 6.

In some embodiments, provided is an aqueous ophthalmic composition comprising about 0.5% w/w to about 1% Compound 1 HCl salt, about 1.5% w/w glycerin, about 0.01% w/w benzalkonium chloride, about 2% w/w polysorbate 80, and about 10 mM citrate buffer in water, and having a pH of about 6.

In some embodiments, provided is an aqueous ophthalmic composition comprising about 0.5% w/w to about 1% Compound 1 HCl salt, about 1.5% w/w glycerin, about 0.01% w/w benzalkonium chloride, about 2% w/w PEG-35 castor oil, and about 10 mM citrate buffer in water, and having a pH of about 6.

In some embodiments, provided is an aqueous ophthalmic composition comprising about 0.5% w/w to about 1% Compound 1 HCl salt, about 1.5% w/w glycerin, about 0.01% w/w benzalkonium chloride, about 2% w/w polysorbate 80, and about 10 mM phosphate buffer in water, and having a pH of about 6.

In some embodiments, provided is an aqueous ophthalmic composition comprising about 0.5% to about 1% w/w Compound 1 HCl salt, about 1.5 w/w glycerin, about 0.01% w/w benzalkonium chloride, about 2% w/w PEG-35 castor oil, and about 10 mM phosphate buffer in water, and having a pH of about 6.

In some embodiments, provided is an aqueous ophthalmic composition comprising about 0.5% w/w Compound 1 HCl salt, about 1.5 w/w glycerin, about 0.01% w/w benzalkonium chloride, about 2% w/w PVP K29/32, and about 10 mM phosphate buffer in water, and having a pH of about 6.

In some embodiments, provided is an aqueous ophthalmic composition comprising about 0.001% to about 2% w/w Compound 1 and/or a cation thereof, about 1% to about 2% w/w of a tonicity modifier, about 0.005% to about 0.02% w/w of a preservative, a buffer, and water, and having a pH of about 5.5 to 7.5.

In some embodiments, provided is an aqueous ophthalmic composition comprising about 0.01% to about 2% w/w Compound 1 and/or a cation thereof, Cl⁻, about 1% to about 2% w/w of a tonicity modifier, about 0.005% to about 0.02% w/w of a preservative, a buffer, and water, and having a pH of about 5.5 to 7.5.

In some embodiments, provided is an aqueous ophthalmic composition comprising about 0.1% to about 2% w/w Compound 1 and/or a cation thereof, Cl⁻, about 1% to about 2% w/w of a tonicity modifier, about 0.005% to about 0.02% w/w of a preservative, a buffer, and water, and having a pH of about 5.5 to 7.5.

In some embodiments, provided is an aqueous ophthalmic composition comprising about 0.1% to about 2% w/w Compound 1 and/or a cation thereof, Cl⁻, about 1% to about 2% w/w glycerin, about 0.005% to about 0.02% w/w benzalkonium chloride, a buffer, and water, and having a pH of about 5.5 to 7.5.

In some embodiments, provided is an aqueous ophthalmic composition comprising about 0.1% to about 2% w/w Compound 1 and/or a cation thereof, Cl⁻, about 1% to about 2% w/w glycerin, about 0.005% to about 0.02% w/w benzalkonium chloride, a buffer, and water, and having a pH of about 6.

In some embodiments, provided is an aqueous ophthalmic composition comprising about 0.1% to about 2% w/w Compound 1 and/or a cation thereof, Cl⁻, about 1.5% w/w glycerin, about 0.005% to about 0.02% w/w benzalkonium chloride, a buffer, and water, and having a pH of about 6.

In some embodiments, provided is an aqueous ophthalmic composition comprising about 0.5% to about 1% w/w Compound 1 and/or a cation thereof, Cl⁻, about 1% to about 2% w/w glycerin, about 0.005% to about 0.02% w/w benzalkonium chloride, a phosphate buffer, and water, having a pH of about 5.5 to 7.5.

In some embodiments, provided is an aqueous ophthalmic composition comprising about 0.5% to 1% w/w Compound 1 and/or a cation thereof, Cl⁻, about 1.5% w/w glycerin, about 0.01% w/w benzalkonium chloride, about 10 mM phosphate buffer, and water, and having a pH of about 6.

In some embodiments, provided is an aqueous ophthalmic composition comprising about 0.5% w/w Compound 1 and/or a cation thereof, Cl⁻, about 1.5% w/w glycerin, about 0.01% w/w benzalkonium chloride, about 10 mM phosphate buffer, and water, and having a pH of about 6.

In some embodiments, provided is an aqueous ophthalmic composition comprising about 1% w/w Compound 1 and/or a cation thereof, Cl⁻, about 1.5% w/w glycerin, about 0.01% w/w benzalkonium chloride, about 10 mM phosphate buffer, and water, and having a pH of about 6.

In some embodiments, provided is an aqueous ophthalmic composition comprising about 0.5% w/w to about 1% Compound 1 and/or a cation thereof, Cl⁻, about 1.5% w/w glycerin, about 0.01% w/w benzalkonium chloride, about 2% w/w polysorbate 80, about 10 mM citrate buffer, and water, and having a pH of about 6.

In some embodiments, provided is an aqueous ophthalmic composition comprising about 0.5% w/w to about 1% Compound 1 and/or a cation thereof, Cl⁻, about 1.5% w/w glycerin, about 0.01% w/w benzalkonium chloride, about 2% w/w PEG-35 castor oil, about 10 mM citrate buffer, and water, and having a pH of about 6.

In some embodiments, provided is an aqueous ophthalmic composition comprising about 0.5% w/w to about 1% Compound 1 and/or a cation thereof, Cl⁻, about 1.5% w/w glycerin, about 0.01% w/w benzalkonium chloride, about 2% w/w polysorbate 80, about 10 mM phosphate buffer, and water, and having a pH of about 6.

In some embodiments, provided is an aqueous ophthalmic composition comprising about 0.5% to about 1% w/w Compound 1 and/or a cation thereof, Cl⁻, about 1.5% w/w glycerin, about 0.01% w/w benzalkonium chloride, about 2% w/w PEG-35 castor oil, about 10 mM phosphate buffer, and water, and having a pH of about 6.

In some embodiments, provided is an aqueous ophthalmic composition comprising about 0.5% w/w Compound 1 and/or a cation thereof, Cl⁻, about 1.5% w/w glycerin, about 0.01% w/w benzalkonium chloride, about 2% w/w PVP K29/32, about 10 mM phosphate buffer, and water, and having a pH of about 6.

In some embodiments, provided is an aqueous ophthalmic composition comprising about 0.5% w/w to about 1% Compound 1 and/or a cation thereof, Cl⁻, about 0.5% w/w NaCl, about 0.01% w/w benzalkonium chloride, about 2% w/w PVP K29/32, about 10 mM phosphate buffer, and water, and having a pH of about 6.

In some embodiments, provided is an aqueous ophthalmic composition comprising about 0.5% w/w to about 1% Compound 1 and/or a cation thereof, Cl⁻, about 0.5% w/w NaCl, about 0.01% w/w benzalkonium chloride, about 2% w/w PVP K29/32, about 10 mM phosphate buffer, and water, and having a pH of about 6.

In some embodiments, provided is an ophthalmic composition comprising about 0.01% to about 1% w/w Compound 1 or a pharmaceutically acceptable salt thereof, about 1% to about 2% w/w of a tonicity modifier, about 0.005% to about 0.02% w/w of a preservative, and a buffer in water, and having a pH of about 5.5 to about 7.5.

In some embodiments, provided is an ophthalmic composition comprising about 0.01% to about 5% w/w Compound 1 HCl salt, about 1% to about 2% w/w glycerin, about 0.005% to about 0.02% w/w benzalkonium chloride, and a buffer in water and having a pH of about 5.5 to about 7.5.

In some embodiments, provided is an ophthalmic composition comprising about 0.1% to 5% w/w Compound 1 HCl salt, about 1.5% w/w glycerin, about 0.01% w/w benzalkonium chloride, and about 10 mM phosphate buffer in water, and having a pH of about 6.

In some embodiments, provided is an aqueous ophthalmic composition comprising about 0.01% to about 5% w/w Compound 1 HCl salt, about 1.5% w/w glycerin, and about 10 mM phosphate buffer in water, and having a pH of about 6. In some embodiments, provided is an aqueous ophthalmic composition comprising about 0.01% to about 5% w/w Compound 1 HCl salt, about 1.5% w/w glycerin, and about 10 mM phosphate buffer in water, and having a pH of about 6, wherein the aqueous ophthalmic composition does not comprise a preservative.

In some embodiments, provided is an aqueous ophthalmic composition prepared by a method comprising mixing about 0.001% to about 2% w/w Compound 1 or a pharmaceutically acceptable salt thereof, about 1% to about 2% w/w of a tonicity modifier, about 0.005% to about 0.02% w/w of a preservative, and a buffer in water. In some embodiments, the method further comprises adjusting the pH to about 5.5 to 7.5.

In some embodiments, provided is an aqueous ophthalmic composition prepared by a method comprising mixing about 0.01% to about 2% w/w Compound 1 or a pharmaceutically acceptable salt thereof, about 1% to about 2% w/w of a tonicity modifier, about 0.005% to about 0.02% w/w of a preservative, and a buffer in water. In some embodiments, the method further comprises adjusting the pH to about 5.5 to 7.5.

In some embodiments, provided is an aqueous ophthalmic composition prepared by a method comprising mixing about 0.1% to about 2% w/w Compound 1 or a pharmaceutically acceptable salt thereof, about 1% to about 2% w/w of a tonicity modifier, about 0.005% to about 0.02% w/w of a preservative, and a buffer in water. In some embodiments, the method further comprises adjusting the pH to about 5.5 to 7.5.

In some embodiments, provided is an aqueous ophthalmic composition prepared by a method comprising mixing about 0.001% to about 2% w/w Compound 1 HCl salt, about 1% to about 2% w/w of a tonicity modifier, about 0.005% to about 0.02% w/w of a preservative, and a buffer in water. In some embodiments, the method further comprises adjusting the pH to about 5.5 to 7.5.

In some embodiments, provided is an aqueous ophthalmic composition prepared by a method comprising mixing about 0.01% to about 2% w/w Compound 1 HCl salt, about 1% to about 2% w/w of a tonicity modifier, about 0.005% to about 0.02% w/w of a preservative, and a buffer in water. In some embodiments, the method further comprises adjusting the pH to about 5.5 to 7.5.

In some embodiments, provided is an aqueous ophthalmic composition prepared by a method comprising mixing about 0.1% to about 2% w/w Compound 1 HCl salt, about 1% to about 2% w/w of a tonicity modifier, about 0.005% to about 0.02% w/w of a preservative, and a buffer in water. In some embodiments, the method further comprises adjusting the pH to about 5.5 to 7.5.

In some embodiments, provided is an aqueous ophthalmic composition prepared by a method comprising mixing about 0.1% to about 2% w/w Compound 1 HCl salt, about 1% to about 2% w/w glycerin, about 0.005% to about 0.02% w/w benzalkonium chloride, and a buffer in water. In some embodiments, the method further comprises adjusting the pH to about 5.5 to 7.5.

In some embodiments, provided is an aqueous ophthalmic composition prepared by a method comprising mixing about 0.1% to about 2% w/w Compound 1 HCl salt, about 1% to about 2% w/w glycerin, about 0.005% to about 0.02% w/w benzalkonium chloride, and a buffer in water. In some embodiments, the method further comprises adjusting the pH to about 6.

In some embodiments, provided is an aqueous ophthalmic composition prepared by a method comprising mixing about 0.1% to about 2% w/w Compound 1 HCl salt, about 1.5% w/w glycerin, about 0.005% to about 0.02% w/w benzalkonium chloride, and a buffer in water. In some embodiments, the method further comprises adjusting the pH to about 6.

In some embodiments, provided is an aqueous ophthalmic composition prepared by a method comprising mixing about 0.5% to about 1% w/w Compound 1 HCl salt, about 1% to about 2% w/w glycerin, about 0.005% to about 0.02% w/w benzalkonium chloride, and a phosphate buffer in water. In some embodiments, the method further comprises adjusting the pH to about 5.5 to 7.5.

In some embodiments, provided is an aqueous ophthalmic composition prepared by a method comprising mixing about 0.5% to 1% w/w Compound 1 HCl salt, about 1.5% w/w glycerin, about 0.01% w/w benzalkonium chloride, and about 10 mM phosphate buffer in water. In some embodiments, the method further comprises adjusting the pH to about 6.

In some embodiments, provided is an aqueous ophthalmic composition prepared by a method comprising mixing about 0.5% w/w Compound 1 HCl salt, about 1.5% w/w glycerin, about 0.01% w/w benzalkonium chloride, and about 10 mM phosphate buffer in water. In some embodiments, the method further comprises adjusting the pH to about 6.

In some embodiments, provided is an aqueous ophthalmic composition prepared by a method comprising mixing about 1% w/w Compound 1 HCl salt, about 1.5% w/w glycerin, about 0.01% w/w benzalkonium chloride, and about 10 mM phosphate buffer in water. In some embodiments, the method further comprises adjusting the pH to about 6.

In some embodiments, provided is an aqueous ophthalmic composition prepared by a method comprising mixing about 0.5% w/w to about 1% Compound 1 HCl salt, about 1.5% w/w glycerin, about 0.01% w/w benzalkonium chloride, about 2% w/w polysorbate 80, and about 10 mM citrate buffer in water. In some embodiments, the method further comprises adjusting the pH to about 6.

In some embodiments, provided is an aqueous ophthalmic composition prepared by a method comprising mixing about 0.5% w/w to about 1% Compound 1 HCl salt, about 1.5% w/w glycerin, about 0.01% w/w benzalkonium chloride, about 2% w/w PEG-35 castor oil, and about 10 mM citrate buffer in water. In some embodiments, the method further comprises adjusting the pH to about 6.

In some embodiments, provided is an aqueous ophthalmic composition prepared by a method comprising mixing about 0.5% w/w to about 1% Compound 1 HCl salt, about 1.5 w/w glycerin, about 0.01% w/w benzalkonium chloride, about 2% w/w polysorbate 80, and about 10 mM phosphate buffer in water. In some embodiments, the method further comprises adjusting the pH to about 6.

In some embodiments, provided is an aqueous ophthalmic composition prepared by a method comprising mixing about 0.5% to about 1% w/w Compound 1 HCl salt, about 1.5 w/w glycerin, about 0.01% w/w benzalkonium chloride, about 2% w/w PEG-35 castor oil, and about 10 mM phosphate buffer in water. In some embodiments, the method further comprises adjusting the pH to about 6.

In some embodiments, provided is an aqueous ophthalmic composition prepared by a method comprising mixing about 0.5% w/w Compound 1 HCl salt, about 1.5% w/w glycerin, about 0.01% w/w benzalkonium chloride, about 2% w/w PVP K29/32, and about 10 mM phosphate buffer in water. In some embodiments, the method further comprises adjusting the pH to about 6.

The compositions described here can be contained in an appropriate sized container (such as up to about 0.1 mL, about 0.2 mL, about 0.3 mL, about 0.4 mL, about 0.5 mL, about 1 mL, about 2.5 mL, about 5 mL, 7.5 mL, about 10 mL, about 15 mL, about 20 mL, about 25 mL, about 30 mL, about 35 mL, or about 40 mL). In some embodiments, the container is suitable for applying eyedrops.

In some embodiments, the container is an appropriate size to contain 1 to 2 unit doses. In some embodiments, the container is sealed. In some embodiments, the container provides an antiseptic seal. In some embodiments, the container comprises a puncture seal. In some embodiments, the container comprises a blow-fill-seal type closure.

In some embodiments, the aqueous ophthalmic compositions of the present disclosure can be prepared by mixing appropriate amounts of Compound 1 or a pharmaceutically acceptable salt thereof, the tonicity modifier, the preservative, and optionally the stabilizer in an aqueous buffer as described herein to form a clear solution.

In some embodiments, provided is a method of treating an ophthalmic disease described herein, comprising administering to an eye of a patient in need of a therapeutically effective amount of an aqueous ophthalmic composition described herein. A single administration or unit dose of the aqueous ophthalmic compositions described herein refers to the application of the composition to one or both eyes, and may comprise one drop, two drops, three drops, four drops, five drops, six drops, seven drops, eight drops or more of the composition each time. The composition may be administered once a day, twice a day, three times a day, four times a day or more frequently at appropriate intervals throughout the day, or as needed. In some embodiments, the composition may be administered by a sustained release delivery system.

In some embodiments, the therapeutically effective amount of the aqueous ophthalmic composition comprises about 0.001 mg to about 10 mg, about 0.01 mg to about 10 mg, about 0.1 mg to about 10 mg, about 0.15 mg to about 8 mg, about 0.25 mg to about 5 mg, or about 1 mg to about 5 mg, about 0.2 mg to about 1 mg, about 0.2 mg to about 0.7 mg, about 0.2 mg to about 0.5 mg of Compound 1 or a pharmaceutically acceptable salt thereof administered to one eye in need of the treatment. In some embodiments, the therapeutically effective amount of the aqueous ophthalmic composition is about 0.01 mL to about 0.5 mL administered to one eye in need of the treatment. In some embodiments, the therapeutically effective amount of the aqueous ophthalmic composition is about 0.005 mL, about 0.01 mL, about 0.025 mL, about 0.03 mL, about 0.035 mL, about 0.04 mL, about 0.05 mL, about 0.07 mL, about 0.1 mL, about 0.15 mL, about 0.2 mL, about 0.25 mL, about 0.3 mL, about 0.35 mL, about 0.4 mL, about 0.45 mL, about 0.5 mL, or any range between any two of the values (end points inclusive), administered to one eye in need of the treatment. In some embodiments, the therapeutically effective amount of the aqueous ophthalmic composition is one to ten drops administered to one or both eyes. In some embodiments, the therapeutically effective amount of the aqueous ophthalmic composition is one drop, two drops, three drops, four drops, five drops, six drops, seven drops, eight drops, nine drops, or ten drops, or any range between any two of the values (end points inclusive), administered to one eye. In some embodiments, the aqueous ophthalmic composition is administered once a day, twice a day, three times a day, four times a day or more frequently at appropriate intervals throughout the day, or as needed.

In some embodiments, provided herein is use of the pharmaceutical compositions described herein in the treatment of ophthalmic diseases described herein. In some embodiments, the aqueous ophthalmic composition is for administration at about 0.005 mL to about 0.5 mL to one eye. In some embodiments, the aqueous ophthalmic composition is for administration at about 0.01 mL, about 0.025 mL, about 0.03 mL, about 0.035 mL, about 0.04 mL, about 0.045 mL, about 0.05 mL, 0.07 mL, about 0.1 mL, about 0.15 mL, about 0.2 mL, about 0.25 mL, about 0.3 mL, about 0.35 mL, about 0.4 mL, about 0.45 mL, or about 0.5 mL, or any range between any two of the values (end points inclusive), to one eye. In some embodiments, one to ten drops of the aqueous ophthalmic composition are administered to one eye. In some embodiments, one drop, two drops, three drops, four drops, five drops, six drops, seven drops, eight drops, nine drops, or ten drops, or any range between any two of the values (end points inclusive) of the aqueous ophthalmic composition are administered to one or both eyes. In some embodiments, the aqueous ophthalmic composition is for administration once a day, twice a day, three times a day, four times a day or more frequently at appropriate intervals throughout the day, or as needed.

In some embodiments, provided is a kit comprising an ophthalmic composition described herein contained within a container prepared from a pharmaceutically acceptable packaging material. Pharmaceutically acceptable packaging materials include but are not limited to low density polyethylene (“LDPE”), high density polyethylene (“HDPE”), polypropylene, polystyrene, polycarbonate, polyesters (such as polyethylene terephthalate and polyethylene naphthalate), nylon, poly(vinyl chloride), poly(vinylidine chloride), poly(tetrafluoroethylene) and other materials known to those of ordinary skill in the art. In some embodiments, the container is a flexible bottle prepared from LDPE or HDPE. In some embodiments, the container contains about 0.01 mL to about 50 mL of the ophthalmic composition, such as about 0.05 mL, about 0.1 mL, about 0.2 mL, about 0.3 mL, about 0.4 mL, about 0.5 mL, about 1 mL, about 2 mL, about 2.5 mL, about 3 mL, about 4 mL, about 5 mL, about 7.5 mL, about 10 mL, about 15 mL, about 20 mL, about 25 mL, about 30 mL, about 35 mL, about 40 mL, about 45 mL, or about 50 mL, or any range between any two of the values (end points inclusive) of the ophthalmic composition. In some embodiments, the container contains multiple doses. In some embodiments, the container contains a single unit dose or daily doses. In some embodiments, the container is sealed. Specialized multi-dose preservative free containers such as those having filters, tips, or dual chamber containers, may also be used.

### EXAMPLES

### Example 1. Preparation of Aqueous Ophthalmic Compositions

In general, the aqueous ophthalmic compositions descried herein are prepared by dissolving excipients in a target volume of water in a suitable container, followed by addition of the active ingredient. After that, pH is adjusted to the desired value and more water is added to the target final volume. The concentrations or amount listed in the examples below may vary by ±10%, ±5% or ±1% of the stated values.

### Example 2. Dissolution Studies

A dissolution study was performed in which the HCl salt or the free base were added to the following formulation buffer at a concentration of 10 mg/mL (1%):
pH 6, 10 mM citrate buffer, 1% PEG400, 1% Glycerin, 0.3% NaCl, 0.05% PS80.

The pH was increased for soluble samples to better understand their properties. The appearance of the samples was visually observed for clarity or cloudiness. The HCl salt was soluble even at a pH of 6, however precipitates formed as pH was increased from 6. The free base was not soluble.

The free base was not soluble with various solubilizers. The only sample which showed a solubility of 1-10 mg/mL was the combination of 3% Glycerin, 5% PEG 400, 5% PEG-35 castor oil, 15% polysorbate 60, 4% polysorbate 80, 2% PVP K29/32, 10% propylene glycol. The 5% PEG 400 with pH adjustment/buffering yielded a clear solution, however the pH was 3. The 10% propylene glycol with pH adjustment/buffering yielded a clear solution with a pH of 6.1, however the buffer concentration for 1 mg/mL Compound 1 was about 20 mM phosphate, and for 10 mg/mL Compound 1 was estimated to be about 200 mM.

TABLE 2-1
Free base solubilization results

| Sample # | Solubilizer description | Solubility result | |
|---|---|---|---|
| 1-1 | 10% propylene glycol | <1 | mg/mL ** |
| 1-2 | 2% PVP K29/32 | <1 | mg/mL |
| 1-3 | 5% PEG-35 castor oil | <1 | mg/mL |
| 1-4 | 5% PEG 400 | <1 | mg/mL * |
| 1-5 | 4% polysorbate 80 | <1 | mg/mL |
| 1-6 | 3% glycerin, 5% PEG 400, 5% PEG-35 | 2-5 | mg/mL |
| | castor oil, 15% polysorbate 60, | | |
| | 4% polysorbate 80, 2% PVP K29/32, | | |
| | 10% propylene glycol | | |

* 1M citric acid added to PEG 400 solubilized the API after 300 μL was added; final pH was 3.0.
** 1M sodium phosphate monobasic added to propylene glycol solubilized the API after 500 μL was added; final pH was 6.1.

The formulations in Table 2-2 were prepared and examined for appearance. Formulations yielding clear solutions were placed at ambient temperature (15° C. to 25° C.) for storage. After 8 weeks, those formulations that remained clear were analyzed by a qualified HPLC method. The pH and osmolality were taken initially (T0) and at 8 weeks (T8W) for those formulations that were clear at T0 and remained clear after 8 weeks of storage. (See Tables 2-3 and 2-4).

All solutions containing NaCl as the tonicity modifier (Formulations 2-4, 2-5, 2-6, 2-10, 2-11, and 2-12) failed to form clear solutions at the beginning or failed to maintain clear solutions after 8 weeks. Furthermore, the formulation containing citrate buffer and PVP K29/32 (Formulation 2-3) produced a cloudy suspension. Most solutions with glycerol were clear through 8 weeks.

TABLE 2-2
Formulations

| | | | | Tonicity | |
|---|---|---|---|---|---|
| Formulation | API | Buffer | Preservative | Modifier | Stabilizer |
| 2-1 | 1% HCl salt | 10 mM citrate, pH 6 | 0.01% BAC | 1.5% | 2% PS80 |
| | | | | glycerol | |
| 2-2 | 1% HCl salt | 10 mM citrate, pH 6 | 0.01% BAC | 1.5% | 2% PEG-35 |
| | | | | glycerol | Castor Oil |
| 2-3 | 1% HCl salt | 10 mM citrate, pH 6 | 0.01% BAC | 1.5% | 2% PVP |
| | | | | glycerol | K29/32 |
| 2-4 | 1% HCl salt | 10 mM citrate, pH 6 | 0.01% BAC | 0.5% | 2% PS80 |
| | | | | NaCl | |
| 2-5 | 1% HCl salt | 10 mM citrate, pH 6 | 0.01% BAC | 0.5% | 2% PEG-35 |
| | | | | NaCl | Castor Oil |
| 2-6 | 1% HCl salt | 10 mM citrate, pH 6 | 0.01% BAC | 0.5% | 2% PVP |
| | | | | NaCl | K29/32 |
| 2-7 | 1% HCl salt | 10 mM phosphate, pH 6 | 0.01% BAC | 1.5% | 2% PS80 |
| | | | | glycerol | |
| 2-8 | 1% HCl salt | 10 mM phosphate, pH 6 | 0.01% BAC | 1.5% | 2% PEG-35 |
| | | | | glycerol | Castor Oil |
| 2-9 | 1% HCl salt | 10 mM phosphate, pH 6 | 0.01% BAC | 1.5% | 2% PVP |
| | | | | glycerol | K29/32 |
| 2-10 | 1% HCl salt | 10 mM phosphate, pH 6 | 0.01% BAC | 0.5% | 2% PS80 |
| | | | | NaCl | |
| 2-11 | 1% HCl salt | 10 mM phosphate, pH 6 | 0.01% BAC | 0.5% | 2% PEG-35 |
| | | | | NaCl | Castor Oil |
| 2-12 | 1% HCl salt | 10 mM phosphate, pH 6 | 0.01% BAC | 0.5% | 2% PVP |
| | | | | NaCl | K29/32 |

TABLE 2-3
Appearance, pH, and Osmolality results (T0)

| Formu- | | pH | Osmolality |
|---|---|---|---|
| lation | Appearance | (T0) | (T0) |
| 2-1 | clear solution | 6.1 | 317 mOsm/kg |
| 2-2 | clear solution | 6.19 | 318 mOsm/kg |
| 2-3 | Cloudy suspension | Not tested - Lack of | |
| 2-4 | Precipitates formed then aggregated/ | clear solution | |
| | flocculated | | |
| 2-5 | Precipitates formed then aggregated/ | | |
| | flocculated | | |
| 2-6 | Precipitates formed then aggregated/ | | |
| | flocculated | | |
| 2-7 | clear solution | 6.17 | 300 mOsm/kg |
| 2-8 | clear solution | 6.18 | 305 mOsm/kg |
| 2-9 | clear solution | 6.19 | 305 mOsm/kg |
| 2-10 | Precipitates formed then solution gelled | Not tested - Lack of | |
| 2-11 | Precipitates formed then solution gelled | clear solution | |
| 2-12 | Gelled after filtration, no flocculated | 6.1 | 287 mOsm/kg |
| | particles but does appear to have started | | |
| | to precipitate | | |

TABLE 2-4
Appearance, pH, and Osmolality results (T8 W)

| Formu- | | pH | Osmolality |
|---|---|---|---|
| lation | Appearance | (T8 W) | (T8 W) |
| 2-1 | clear solution | 5.99 | 320 mOsm/kg |
| 2-2 | clear solution | 6.13 | 318 mOsm/kg |
| 2-3 | Cloudy suspension | Not tested - Lack of | |
| 2-4 | Precipitates formed then aggregated/ | clear solution | |
| | flocculated | | |
| 2-5 | Precipitates formed then aggregated/ | | |
| | flocculated | | |
| 2-6 | Precipitates formed then aggregated/ | | |
| | flocculated | | |
| 2-7 | clear solution | 6.00 | 302 mOsm/kg |
| 2-8 | clear solution | 6.08 | 305 mOsm/kg |
| 2-9 | clear solution | 6.13 | 306 mOsm/kg |
| 2-10 | Precipitates formed then solution | Not tested - Lack of | |
| | gelled | clear solution | |
| 2-11 | Precipitates formed then solution | | |
| | gelled | | |
| 2-12 | Precipitates present in solution | | |

As shown in Table 2-4, Formulations 2-1, 2-2, 2-7, 2-8, and 2-9 remained clear after 8 weeks. Those formulations were tested for impurities and mass balance via HPLC analysis. Formulation 7 had two impurity peaks that were deemed significant when compared to the other formulations.

### Example 3. In Vivo Compatibility Study

An in vivo study was conducted to test four of the formulations in Table 3-1 for tolerability in rabbits. In this study, each group consisted of 3 rabbits (2 males, 1 female) receiving one formulation. The rabbits were dosed 4 times a day (doses were separated by 2 hours) for 7 consecutive days. Dosing consisted of administration (topical ocular instillation) of 50 μL of the appropriate formulation into the right eye of the rabbit; the left eye served as the contralateral control and remained untreated. The eyes of each rabbit were graded for signs of irritation using the Draize scoring method, prior to treatment and again on a daily basis after the last daily administration. A score of 0 indicates that there was no irritation noted, whereas a score of 1, 2, 3, or 4 indicates that some irritation was observed. The higher the number, the more severe the irritation that was observed.

Each formulation and category has a maximum of 21 observations (3 rabbits×7 days). The tolerability scores are presented in Table 3-2. The solutions with citrate buffer or phosphate buffer and PEG-35 Castor Oil (Formulations 3-2 and 3-3) were the best tolerated among the four formulations.

TABLE 3-1
Formulations in tolerability study

| | | | | Tonicity | |
|---|---|---|---|---|---|
| Formulation | API | Buffer | Preservative | Modifier | Stabilizer |
| 3-1 | 1.0% HCl salt | 10 mM citrate, pH 6 | 0.01% BAC | 1.5% | 2% PS80 |
| | | | | glycerol | |
| 3-2 | 1.0% HCl salt | 10 mM citrate, pH 6 | 0.01% BAC | 1.5% | 2% PEG-35 |
| | | | | glycerol | castor oil |
| 3-3 | 1.0% HCl salt | 10 mM phosphate, pH 6 | 0.01% BAC | 1.5% | 2% PEG-35 |
| | | | | glycerol | castor oil |
| 3-4 | 1.0% HCl salt | 10 mM phosphate, pH 6 | 0.01% BAC | 1.5% | 2% PVP |
| | | | | glycerol | K29/32 |

TABLE 3-2
Irritation scores from tolerability study

| | Cornea | | | | | Iris | | | Redness | | | | Chemosis | | | | | Discharge | | | |
|---|---|---|---|---|---|---|---|---|---|---|---|---|---|---|---|---|---|---|---|---|---|
| Formulation | 0 | 1 | 2 | 3 | 4 | 0 | 1 | 2 | 0 | 1 | 2 | 3 | 0 | 1 | 2 | 3 | 4 | 0 | 1 | 2 | 3 |
| 3-1 | 21 | 0 | 0 | 0 | 0 | 21 | 0 | 0 | 13 | 8 | 0 | 0 | 21 | 0 | 0 | 0 | 0 | 10 | 10 | 1 | 0 |
| 3-2 | 21 | 0 | 0 | 0 | 0 | 21 | 0 | 0 | 21 | 0 | 0 | 0 | 21 | 0 | 0 | 0 | 0 | 21 | 0 | 0 | 0 |
| 3-3 | 21 | 0 | 0 | 0 | 0 | 21 | 0 | 0 | 21 | 0 | 0 | 0 | 21 | 0 | 0 | 0 | 0 | 21 | 0 | 0 | 0 |
| 3-4 | 13 | 2 | 6 | 0 | 0 | 21 | 0 | 0 | 5 | 5 | 8 | 3 | 13 | 2 | 6 | 0 | 0 | 8 | 4 | 9 | 0 |

### Example 4. Stability

Solutions were prepared with different levels of PEG-35 castor oil (2%, 1%, and 0%) (Table 4-1) and held at both the long term storage condition of 25° C. and an accelerated storage condition of 40° C. After one month all samples were analyzed by a qualified HPLC method.

TABLE 4-1
Stabilizer impact study formulations

| | | | | Tonicity | |
|---|---|---|---|---|---|
| Formulation | API | Buffer | Preservative | Modifier | Stabilizer |
| 4-1 | 0.5% HCl salt | 10 mM phosphate, pH 6 | 0.1% BAC | 1.5% | 2% PEG-35 |
| | | | | glycerol | castor oil |
| 4-2 | 0.5% HCl salt | 10 mM phosphate, pH 6 | 0.1% BAC | 1.5% | 1% PEG-35 |
| | | | | glycerol | castor oil |
| 4-3 | 0.5% HCl salt | 10 mM phosphate, pH 6 | 0.1% BAC | 1.5% | 0% PEG-35 |
| | | | | glycerol | castor oil |

TABLE 4-2
Impurity result summary - 1 Month

| Formulation | Total impurities @ 1 M | | Mass Balance @ 1 M | |
|---|---|---|---|---|
| 4-1 | 25° C. | 2.44% | 25° C. | 111.04% |
| | 40° C. | 4.70% | 40° C. | 106.27% |
| 42 | 25° C. | 2.04% | 25° C. | 114.07% |
| | 40° C. | 2.99% | 40° C. | 111.32% |
| 4-3 | 25° C. | 1.58% | 25° C. | 113.08% |
| | 40° C. | 1.69% | 40° C. | 110.63% |

A formulation with increased API concentration and decreased preservative concentration was found to be stable at 25° C. for at least 6 months.

| | | | Tonicity |
|---|---|---|---|
| API | Buffer | Preservative | Modifier |
| 1.0% HCl salt | 10 mM phosphate, pH 6 | 0.01% BAC | 1.5% glycerol |

### Example 5. Toxicity Studies in Dogs and Rabbits

Toxicity studies were conducted for the following two formulations in dogs and rabbits.

| | | | | Tonicity |
|---|---|---|---|---|
| Formulation | API | Buffer | Preservative | Modifier |
| 5-1 | 1.0% | 10 mM phosphate, | 0.01% | 1.5% |
| | HCl salt | pH 6 | BAC | glycerol |
| 5-2 | 0.5% | 10 mM phosphate, | 0.01% | 1.5% |
| | HCl salt | pH 6 | BAC | glycerol |

Study 1: Each formulation was administered to 7 dogs of each sex with 4 dogs per sex being used for the toxicity evaluation and 3 dogs per sex were used as recovery animals to evaluate the reversibility of potential treatment-related effects. Each dog received 4 doses a day of 50 μL of the designated formulation for 28 days (recovery dogs were held with no treatment for an additional 14 days). No treatment-related deaths or clinical signs of toxicity were noted in the study.

Study 2: Each formulation was administered to 11 rabbits of each sex. The rabbits were split with 5 rabbits per sex being used for the core toxicity, and 3 rabbits per sex were used for assessing toxicokinetics, and 3 rabbits per sex were used as recovery animals to evaluate the reversibility of potential treatment-related effects. Each rabbit received 4 doses a day of 50 Compound 1 ophthalmic solution for 28 days (recovery rabbits were held with no treatment for an additional 14 days). No treatment-related deaths or clinical signs of toxicity were noted in the study.

### Example 6. Compound 1 in a Murine Model of Allergic Conjunctivitis

Compound 1 showed efficacy in relief of allergic conjunctivitis in a murine model of the disease.

Mice were sensitized with subcutaneously injected short ragweed allergen (day 1, day 11), and underwent conjunctival allergen challenge (CAC) on day 18 with short ragweed allergen. Following challenge, the mice were randomized into treatment groups (n=8) and given topical test compounds: vehicle, 0.1% Compound 1, 1% Compound 1, 1% prednisolone, or saline (BSS) twice a day for 2 days, and three times a day for an additional 4 days during allergen challenges. Challenges were conducted twice daily, on day 21 thru 24. Responses to allergen challenge were evaluated after challenges 1, 4, 6 and 8.

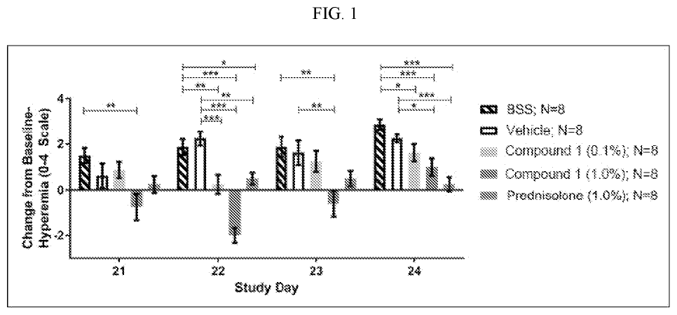

As exemplified in FIG. 1, Compound 1 (1%) significantly reduced mean change in hyperemia (pre-versus post-CAC) compared to saline on 4 of 4 test days; Compound 1 (0.1%) significantly reduced the mean change in hyperemia compared to saline on 2 of 4 test days. The positive control prednisolone (1.0%) also elicited a significant decrease in the mean change in hyperemia on 2 of 4 test days. A statistically significant reduction in overall mean hyperemia was observed with 1% Compound 1 on 3 of 4 test days. No significant changes were seen for ocular discharge, lid swelling, or squinting.

### Example 7. Clinical Efficacy and Safety of Compound 1 for the Treatment of Acute and Chronic Allergic Conjunctivitis

In a study utilizing the clinical model of the conjunctival allergen challenge (CAC) qualifying patients with acute or chronic allergic conjunctivitis were randomized to receive one of the five interventions listed below, each was given six doses at 1 drop per dose. The CAC model is a standardized clinical methodology for evaluation of novel drugs for allergy and anti-inflammatory activity, accepted by the FDA for clinical development of novel therapeutics, and has been used for development of 19 drugs currently on the market. In the CAC model, doses of allergen are administered in a controlled fashion to patients eyes in the office setting to induce a controlled allergic reaction. Drug is administered at pre-specified timepoints and signs and symptoms are collected also at pre-specified timepoints to assess efficacy.

| Arms | Assigned Interventions | n |
|---|---|---|
| Compound 1: 0.5% | Drug: aqueous ophthalmic Composition A | 32 |
| Compound 1: 1% | Drug: aqueous ophthalmic Composition B | 29 |
| Placebo Comparator: | Drug: aqueous ophthalmic composition | 29 |
| Compound 1 0% | comprising 0% Compound 1 | |
| Patanol ® | during acute and chronic phases | 15 |
| Patanol ®/Pred | Patanol ® during acute phase/Pred forte ® | 15 |
| forte ® | during chronic phase | |

Composition A: 0.5% w/w Compound 1 HCl salt, 1.5% w/w glycerin, 0.01% w/w BAC in 10 mM phosphate buffer, pH 6.0.
Composition B: 1.0% w/w Compound 1 HCl salt, 1.5% w/w glycerin, 0.01% w/w BAC in 10 mM phosphate buffer, pH 6.0.
Placebo: 0.01% w/w BAC, 1.5% w/w glycerol, pH 6.0.
   After receiving screening and baseline CACs, patients were assigned in a masked randomization fashion to receive a study treatment at 8 hour or 15 min pre-CAC at Visits 4b and 5, respectively. Patients then continued dosing BID and received a series of repeat CAC challenges, 6 and 8 hrs following dosing.

### Outcome Measures:

Ocular Itching [Time Frame: 5, 7, and 10 minutes post allergen administration (CAC)] Ocular Itching were assessed by the subjects using a 0-4 scale (0=none to 4=severe). Average of ocular itching score over both eyes were analyzed.
Conjunctival Redness [Time Frame: 7, 15, and 20 minutes post allergen administration CAC]
Conjunctival Redness were assessed by the investigator using a 0-4 scale (0=none to 4=severe). Average of conjunctival redness score over both eyes were analyzed.
Other variables assessed for efficacy include lid swelling, chemosis, tearing, nasal symptoms, and indication of biological activity via exploratory assessment of biomarkers, and exploratory imaging of inflammation with in vivo confocal microscopy.

### Results:

Both concentrations of Compound 1 showed clear and consistent biological effect in this study.

Both Compositions A and B showed statistically superior clinically relevant treatment effect on ocular redness and conjunctival swelling (chemosis) to vehicle and Patanol® when administered 8 hours prior to challenge. In addition, both Compositions A and B showed efficacy in preventing ciliary redness and episcleral redness when administered 8 hours prior to CAC. Differences were statistically significant from Vehicle (all 3 parameters) and Patanol® (ciliary redness and episcleral redness).

Table 7-1 shows the conjunctival redness scores of subjects treated with Compositions A, B or Patanol® as compared to Vehicle evaluated by the investigators at Visits 4b (allergen challenge was given 8 hours after the first dose) and 5a (allergen challenge was given 15 minutes after the second dose). At Visits 4b and 5a all patients in the Patanol® and Patanol®/Pred Forte® groups received Patanol®.

A larger negative number indicates more efficacy (i.e. greater difference between drug and placebo). Surprisingly, at Visit 4b which measures efficacy 8 hours after dosing (duration of action), Compositions A and B performed superior compared with standard allergy therapy, Patanol® which is currently a leading drug on the market and was included as an active comparator. Also surprising is the data suggests that Compound 1 works better at 8 hours after dosing, as compared with 15 minutes after dosing (Visit 5a).

TABLE 7-1
Conjunctival Redness

| | Time | Composition A | Composition B | Patanol ® |
|---|---|---|---|---|
| Visit | point | (N = 32) | (N = 29) | (N = 30) |
| 4b | 7 | −0.62 ^{a} | −0.52 ^{a} | −0.38 ^{a} |
| | 15 | −0.72 ^{a, b} | −0.67 ^{a, b} | −0.38 ^{a} |
| | 20 | −0.81 ^{a, b} | −0.62 ^{a, b} | −0.33 ^{a} |
| 5a | 7 | −0.22 | 0.02 | −0.76 ^{a} |
| | 15 | −0.05 | 0.16 | −0.42 ^{a} |
| | 20 | −0.19 | 0.10 | −0.55 ^{a} |

In Tables 7-1 to 7-11, a: statistically significant difference from Vehicle; b: statistically significant superiority to Patanol®

Tables 7-2 to 7-11 show the effects of Compositions A, B or Patanol® as compared to Vehicle at Visits 4b and 5a on ciliary redness, episcleral redness, chemosis, eyelid swelling, tearing, rhinorrhea, nasal pruritus, ear or palate pruritus, nasal congestion and ocular itching.

TABLE 7-2
Ciliary Redness

| | Time | Composition A | Composition B | Patanol ® |
|---|---|---|---|---|
| Visit | point | (N = 32) | (N = 29) | (N = 30) |
| 4b | 7 | −0.65 ^{a} | −0.62 ^{a} | −0.34 ^{a} |
| | 15 | −0.76 ^{a, b} | −0.67 ^{a} | −0.37 ^{a} |
| | 20 | −0.84 ^{a, b} | −0.66 ^{a, b} | −0.31 |
| 5a | 7 | −0.19 | 0.11 | −0.87 ^{a} |
| | 15 | −0.10 | 0.21 | −0.50 ^{a} |
| | 20 | −0.18 | 0.13 | −0.65 ^{a} |

TABLE 7-3
Episcleral Redness

| | Time | Composition A | Composition B | Patanol ® |
|---|---|---|---|---|
| Visit | point | (N = 32) | (N = 29) | (N = 30) |
| 4b | 7 | −0.70 ^{a} | −0.65 ^{a} | −0.42 ^{a} |
| | 15 | −0.83 ^{a} | −0.77 ^{a} | −0.38 ^{a} |
| | 20 | −0.91 ^{a, b} | −0.73 ^{a, b} | −0.38 ^{a} |
| 5a | 7 | −0.22 | 0.02 | −0.80 ^{a} |
| | 15 | −0.08 | 0.06 | −0.51 ^{a} |
| | 20 | −0.21 | 0.03 | −0.59 ^{a} |

TABLE 7-4
Chemosis

| | Time | Composition A | Composition B | Patanol ® |
|---|---|---|---|---|
| Visit | point | (N = 32) | (N = 29) | (N = 30) |
| 4b | 7 | −0.38 ^{a} | −0.26 ^{a} | −0.33 ^{a} |
| | 15 | −0.54 ^{a} | −0.42 ^{a} | −0.44 ^{a} |
| | 20 | −0.50 ^{a} | −0.52 ^{a} | −0.44 ^{a} |
| 5a | 7 | −0.21 | 0.09 | −0.51 ^{a} |
| | 15 | −0.28 | 0.39 | −0.52 ^{a} |
| | 20 | −0.16 | 0.40 | −0.56 ^{a} |

TABLE 7-5
Eyelid Swelling

| | Time | Composition A | Composition B | Patanol ® |
|---|---|---|---|---|
| Visit | point | (N = 32) | (N = 29) | (N = 30) |
| 4b | 7 | −0.07 | −0.19 | −0.45 ^{a} |
| | 15 | 0.11 | −0.07 | −0.35 |
| | 20 | −0.06 | 0.09 | −0.34 |
| 5a | 7 | 0.07 | 0.15 | −0.32 |
| | 15 | 0.16 | 0.13 | −0.39 ^{a} |
| | 20 | 0.15 | 0.26 | −0.26 |

TABLE 7-6
Tearing

| | Time | Composition A | Composition B | Patanol ® |
|---|---|---|---|---|
| Visit | point | (N = 32) | (N = 29) | (N = 30) |
| 4b | 7 | −0.04 | −0.40 | −0.65 ^{a} |
| | 15 | 0.01 | 0.01 | −0.39 |
| | 20 | −0.02 | 0.01 | −0.58 ^{a} |
| 5a | 7 | −0.16 | −0.18 | −0.47 ^{a} |
| | 15 | −0.16 | −0.02 | −0.39 ^{a} |
| | 20 | −0.19 | −0.07 | −0.30 |

TABLE 7-7
Rhinorrhea

| | Time | Composition A | Composition B | Patanol ® |
|---|---|---|---|---|
| Visit | point | (N = 32) | (N = 29) | (N = 30) |
| 4b | 7 | 0.1 | −0.1 | −0.6 ^{a} |
| | 15 | 0.2 | 0.1 | −0.5 ^{a} |
| | 20 | 0.0 | 0.1 | −0.5 ^{a} |
| 5a | 7 | −0.1 | −0.1 | −0.6 ^{a} |
| | 15 | −0.2 | −0.2 | −0.6 ^{a} |
| | 20 | −0.4 | −0.4 ^{a} | −0.8 ^{a} |

TABLE 7-8
Nasal Pruritus

| | Time | Composition A | Composition B | Patanol ® |
|---|---|---|---|---|
| Visit | point | (N = 32) | (N = 29) | (N = 30) |
| 4b | 7 | 0.0 | 0.0 | −0.2 |
| | 15 | −0.2 | 0.0 | −0.7 ^{a} |
| | 20 | −0.2 | 0.0 | −0.5 ^{a} |
| 5a | 7 | −0.2 | −0.3 | −0.5 ^{a} |
| | 15 | −0.4 | −0.4 | −0.7 ^{a} |
| | 20 | −0.5 ^{a} | −0.5 ^{a} | −0.6 ^{a} |

TABLE 7-9
Ear or Palate Pruritus

| | Time | Composition A | Composition B | Patanol ® |
|---|---|---|---|---|
| Visit | point | (N = 32) | (N = 29) | (N = 30) |
| 4b | 7 | 0.1 | 0.1 | −0.6 ^{a} |
| | 15 | −0.1 | 0.0 | −0.6 ^{a} |
| | 20 | 0.3 | 0.1 | −0.2 |
| 5a | 7 | −0.3 | −0.3 | −0.6 ^{a} |
| | 15 | −0.3 | −0.4 | −0.7 ^{a} |
| | 20 | −0.3 | −0.5 | −0.6 ^{a} |

TABLE 7-10
Nasal Congestion

| | Time | Composition A | Composition B | Patanol ® |
|---|---|---|---|---|
| Visit | point | (N = 32) | (N = 29) | (N = 30) |
| 4b | 7 | 0.0 | −0.1 | −0.4 |
| | 15 | 0.0 | 0.0 | −0.7 ^{a} |
| | 20 | −0.1 | −0.1 | −0.6 ^{a} |
| 5a | 7 | −0.4 | 0.0 | −0.6 ^{a} |
| | 15 | −0.6 ^{a} | −0.4 | −0.8 ^{a} |
| | 20 | −0.4 ^{a} | −0.3 | −0.7 ^{a} |

TABLE 7-11
Ocular Itching

| | Time | Composition A | Composition B | Patanol ® |
|---|---|---|---|---|
| Visit | point | (N = 32) | (N = 29) | (N = 30) |
| 4b | 5 | −0.05 | −0.16 | −1.33 ^{a} |
| | 7 | 0.06 | −0.15 | −1.24 ^{a} |
| | 10 | 0.06 | 0.10 | −1.00 ^{a} |
| 5a | 5 | −0.10 | 0.08 | −1.67 ^{a} |
| | 7 | 0.00 | 0.22 | −1.53 ^{a} |
| | 10 | −0.03 | 0.28 | −1.17 ^{a} |

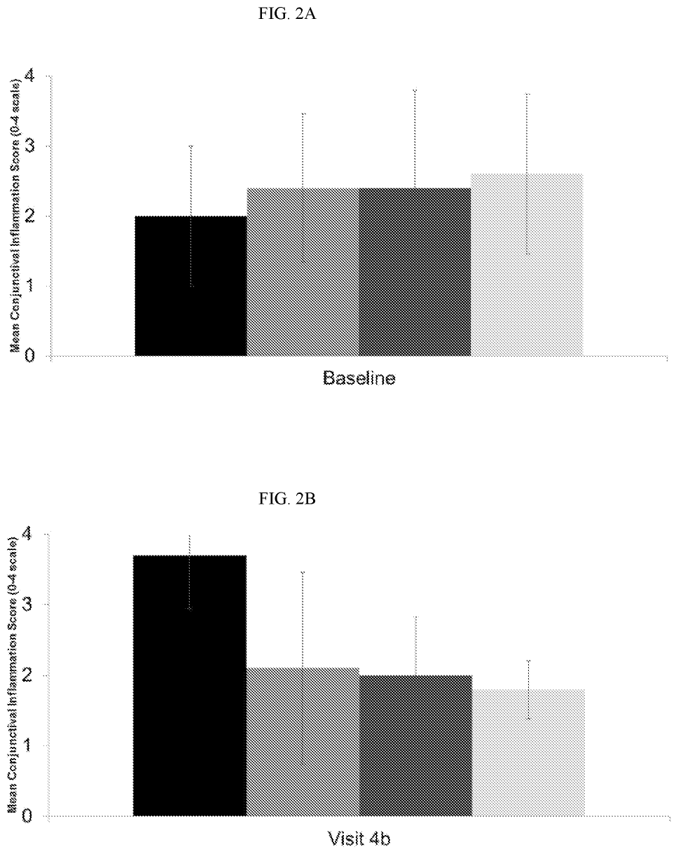

The conjunctiva was imaged using in vivo confocal microscopy to assess the micro vasculature, and to score the inflammation on a scale from 0 (no white blood cells) to 4 (visible inflammation of cells). FIGS. 2A and 2B show the scores at baseline (no treatment) and the scores after CAC which was 8 hours after treatment, respectively. FIG. 2B shows effect of the Compound 1 formulations on reducing objective imaging of inflammation compared with placebo. In the bar graph, the bars represent from left to right; placebo, Patanol, Composition B, Composition A.

This is the first time a specific Syk inhibitor has been evaluated clinically in the eye in this manner. In fact, past attempts at formulating a soluble and stable formulation of a Syk specific inhibitor, and demonstrating efficacy in relevant models, have failed. Given the accepted mechanism of action of Syk kinase involvement in mast cell degranulation, and the central role of the mast cell in the allergic response the clinical data supports the surprising efficacy of Compound 1 in reducing vasodilation (redness), and reducing inflammation. This effect is robust as also shown by reduced chemosis (inflammation of the conjunctival tissue) and objectively via confocal imaging with lower inflammation scores of white blood cells present. Specifically Compound 1 reduced redness to a greater extent at 8 hours following dosing compared with 15 minutes. This is as opposed to current standard drugs such as anti-histamine/mast cell stabilizers (such as Patanol®, the active comparator included in this trial and a market leading drug for approx. two decades for eye allergy) which generally show higher level of efficacy at 15 minute onset compared with duration of action (8 hours in this study). This clinical data supports a robust effect of Compound 1 on reducing hyperemia and inflammation on the eye. It is contemplated that the described compositions surprisingly provide improved comfort, safety, efficacy, solubility, and stability.

Compositions A and B were found to be safe and well tolerated. There were no treatment emergent serious adverse events and all treatment emergent adverse events in the Compound 1 groups were rated mild. Drop comfort scores of Compound 1 compositions were rated on the comfortable part of the scale and within the historical average of currently marketed products.

What is claimed is:

1. An ophthalmic composition comprising about 0.001% w/w to about 10% w/w of Compound 1 of the formula: 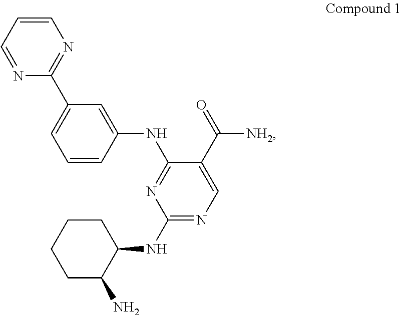 [SMILES: NC(=O)c1cnc(N[C@@H]2CCCC[C@@H]2N)nc1Nc1cccc(-c2ncccn2)c1]
or a pharmaceutically acceptable salt thereof.

2. An ophthalmic composition comprising about 0.01% w/w to about 10% w/w of Compound 1 or a pharmaceutically acceptable salt thereof, a tonicity modifier, a buffer and water.

3. An ophthalmic composition comprising about 0.1% w/w to about 1% w/w of Compound 1 or a pharmaceutically acceptable salt thereof, a tonicity modifier, a buffer and water.

4. The ophthalmic composition of any one of claims 1-3, comprising Compound 1 HCl salt.

5. The ophthalmic composition of any one of claims 1-4, comprising about 0.1% to about 5% of a tonicity modifier, about 0.005 to about 0.02% w/w of a preservative, and a buffer, and having a pH of about 5.5 to 7.

6. The ophthalmic composition of any one of claims 1-4, comprising about 0.2% to about 2% w/w of a tonicity modifier, about 0.005 to about 0.02% w/w of a preservative, and a buffer, and having a pH of about 6.

7. The ophthalmic composition of any one of claims 1-4, comprising about 0.5% to about 1.5% w/w of a tonicity modifier, about 0.01% w/w of a preservative, and a buffer, and having a pH of about 6.

8. The ophthalmic composition of any one of claims 5-7, wherein the tonicity modifier is one or more of glycerin, NaCl, and KCl.

9. The ophthalmic composition of any one of claims 5-7, wherein the tonicity modifier is glycerin.

10. The ophthalmic composition of any one of claims 5-9, wherein the preservative is benzalkonium chloride.

11. The ophthalmic composition of any one of claims 5-10, wherein the buffer is a phosphate buffer.

12. The ophthalmic composition of any one of claims 5-10, wherein the buffer is a citrate buffer.

13. An ophthalmic composition comprising about 0.01% to about 1% w/w Compound 1 or a pharmaceutically acceptable salt thereof, about 1% to about 2% w/w of a tonicity modifier, about 0.005% to about 0.02% w/w of a preservative, and a buffer in water, and having a pH of about 5.5 to about 7.5.

14. An ophthalmic composition comprising about 0.01% to about 5% w/w Compound 1 HCl salt, about 1% to about 2% w/w glycerin, about 0.005% to about 0.02% w/w benzalkonium chloride, and a buffer in water and having a pH of about 5.5 to about 7.5.

15. An ophthalmic composition comprising about 0.1% to about 5% w/w Compound 1 HCl salt, about 1% to about 2% w/w glycerin, about 0.005% to about 0.02% w/w benzalkonium chloride, and a phosphate buffer in water, and having a pH of about 5.5 to 7.5.

16. An ophthalmic composition comprising about 0.1% to 5% w/w Compound 1 HCl salt, about 1.5 w/w glycerin, about 0.01% w/w benzalkonium chloride, and about 10 mM phosphate buffer in water, and having a pH of about 6.

17. An ophthalmic composition comprising about 0.5% w/w Compound 1 HCl salt, about 1.5% w/w glycerin, about 0.01% w/w benzalkonium chloride, and about 10 mM phosphate buffer in water, and having a pH of about 6.

18. An ophthalmic composition comprising about 1% w/w Compound 1 HCl salt, about 1.5% w/w glycerin, about 0.01% w/w benzalkonium chloride, and about 10 mM phosphate buffer in water, and having a pH of about 6.

19. A method of treating an ophthalmic disease or condition comprising administering to an eye of a patient in need thereof a therapeutically effective amount of the ophthalmic composition of any one of claims 1-18.

20. The method of claim 19, wherein the ophthalmic disease or condition is allergic and/or inflammatory, including signs and/or symptoms of one or more of allergic conjunctivitis (including acute allergic conjunctivitis, chronic allergic conjunctivitis, seasonal allergic conjunctivitis or perennial allergic conjunctivitis), rhinoconjunctivis, dry eye, keratoconjunctivitis, blepharitis, dermatitis of the eyelids, blepharoconjunctivitis, ptyergium, post corneal transplant, pingueculitis, episcleritis, scleritis, keratitis, peripheral corneal infiltrate, fungal keratitis, bacterial and viral conjunctivitis, post-operative inflammation, eye inflammation, inflammation of the ocular surface or eyelids, anterior chamber or posterior chamber of the eye, atopic keratoconjunctivitis (AKC), vernal keratoconjunctivitis (VKC), giant papillary conjunctivitis (GPC), neurotrophic keratitis, GVHD-graft versus host disease, traumatic or post-surgical iritis, uveitis, pingueculum, pterygium, contact lens induced dry eye, ocular surface inflammation, irritation, and/or hyperemia, posterior uveitis, retina diseases, diabetic macular edema, branch retinal vein occlusion (BRVO), central retinal vein occlusion (CRVO), eye redness, eyelid swelling, eyelid congestion, swelling eye, itchy eye, steroid-responsive inflammation of the palpebral and bulbar conjunctiva, cornea, and anterior segment of the globe, and ocular conditions for which a corticosteroid is indicated.

21. The method of claim 19, wherein the ophthalmic disease or condition is one or more of dry eye, blepharitis, ptyergium, peripheral corneal infiltrate, post corneal transplant, pingueculitis, episcleritis/scleritis, atopic keratoconjunctivitis, fungal keratitis, allergy, AKC, VKC, GPC, bacterial or viral conjunctivitis, anterior uveitis, traumatic or post-surgical iritis, eyelid swelling, eye redness, irritation, ocular surface inflammation, or post-operative inflammation.

22. The method of claim 19, wherein the ophthalmic disease or condition is chronic allergic conjunctivitis.

23. The method of claim 19, wherein the ophthalmic disease or condition is acute allergic conjunctivitis.

24. The method of any one of claims 19-23, whereby one or more of redness, inflammation, swelling, discomfort, watery eye and itching of the eye, keratitis, corneal staining, conjunctival staining, or markers of inflammation of the eye is reduced or eliminated.

25. A method for treating an ophthalmic disease or condition comprising administering topically to a patient in need thereof a therapeutically effective amount of Compound 1 of the formula: 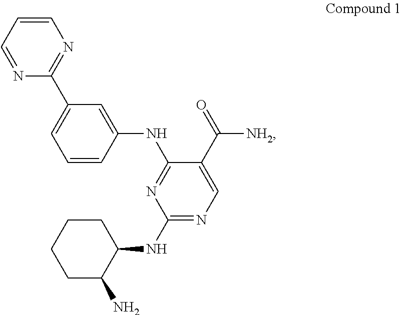 [SMILES: NC(=O)c1cnc(N[C@@H]2CCCC[C@@H]2N)nc1Nc1cccc(-c2ncccn2)c1]
or a pharmaceutically acceptable salt thereof.

26. The method of claim 25, wherein Compound 1 or a pharmaceutically acceptable salt thereof is administered in an amount of about 0.01 mg to about 1 mg to an eye.

27. The method of claim 25 or 26, wherein Compound 1 or a pharmaceutically acceptable salt thereof is administered once a day.

28. The method of claim 25 or 26, wherein Compound 1 or a pharmaceutically acceptable salt thereof is administered twice a day.

29. The method of any one of claims 25-28, wherein the ophthalmic disease or condition is allergic and/or inflammatory, including signs and/or symptoms of one or more of allergic conjunctivitis (including acute allergic conjunctivitis, chronic allergic conjunctivitis, seasonal allergic conjunctivitis or perennial allergic conjunctivitis), irritation, rhinoconjunctivis, dry eye, keratitis, keratoconjunctivitis, blepharitis, blepharoconjunctivitis, ptyergium, post corneal transplant, pingueculitis, episcleritis, scleritis, peripheral corneal infiltrate, fungal keratitis, dermatitis of the eyelids, bacterial or viral conjunctivitis, post-operative inflammation, eye inflammation, inflammation of the ocular surface, eyelids, anterior chamber or posterior chamber of the eye, atopic keratoconjunctivitis (AKC), neurotrophic keratitis, GVHD-graft versus host disease, GPC, vernal keratoconjunctivitis (VKC), viral bacterial fungal or parasitic infection, traumatic and post-surgical iritis, uveitis, pingueculum, pterygium, contact lens induced dry eye, posterior uveitis, anterior uveitis, retina diseases such as macular edema associated with cystoid macular edema, diabetic macular edema, branch retinal vein occlusion (BRVO), central retinal vein occlusion (CRVO), eye redness, eyelid swelling, eyelid congestion, swelling eye, itchy eye, discomfort, keratitis, corneal staining, conjunctival staining, markers of inflammation of the eye, steroid-responsive inflammation of the palpebral and bulbar conjunctiva, cornea, and anterior segment of the globe, and ocular conditions for which a corticosteroid is indicated.

30. The method of any one of claims 25-28, wherein the ophthalmic disease or condition is one or more of dry eye, allergic conjunctivitis, keratoconjunctivitis (sicca), keratitis, blepharitis, ptyergium, peripheral corneal infiltrate, post corneal transplant, pingueculitis, episcleritis/scleritis, atopic keratoconjunctivitis, fungal keratitis, bacterial and viral conjunctivitis, anterior uveitis, or post-operative inflammation, and signs and/or symptoms thereof.

31. The method of any one of claims 25-28, wherein the ophthalmic disease or condition is allergic conjunctivitis.

32. The method of any one of claims 25-28, wherein the ophthalmic disease or condition is acute allergic conjunctivitis.

33. The method of any one of claims 25-28, wherein the ophthalmic disease or condition is chronic allergic conjunctivitis.

34. The method of any one of claims 25-28, wherein the ophthalmic disease or condition is dry eye.

35. The method of any one of claims 25-34, whereby one or more of redness, inflammation, swelling, eyelid congestion, watery eye and itching of the eye is reduced or eliminated.
